(19) United States
(12) Patent Application Publication
(10) Pub. No.: US 2026/0035749 A1
(43) Pub. Date: Feb. 5, 2026

(54) METHODS AND COMPOSITIONS FOR PREDICTING AND/OR MONITORING CARDIOVASCULAR DISEASE AND TREATMENTS THEREFOR

(71) Applicant: Cardio Diagnostics, Inc., Chicago, IL (US)

(72) Inventors: Meeshanthini V. Dogan, Chicago, IL (US); Robert Philibert, Iowa City, IA (US); Timur K. Dogan, Chicago, IL (US)

(21) Appl. No.: 19/359,391

(22) Filed: Oct. 15, 2025

Related U.S. Application Data
(62) Division of application No. 17/466,786, filed on Sep. 3, 2021, now abandoned.
(60) Provisional application No. 63/074,878, filed on Sep. 4, 2020.

(51) Int. Cl. C12Q 1/6883; G16B 20/20

(52) U.S. Cl. CPC C12Q 1/6883; G16B 20/20; C12Q 2600/154; C12Q 2600/156

(57) ABSTRACT

This document describes methods and compositions for predicting cardiovascular disease (CVD). Specifically, this document describes methods and compositions for determining the methylation status of at least one CpG locus and the sequence of at least one single nucleotide polymorphism (SNP) that are predictive for the incidence (e.g., one-year, three-year, five-year incidence) of CVD.

### CROSS REFERENCE TO RELATED APPLICATIONS

This application is a Divisional application of U.S. application Ser. No. 17/466,786 filed on Sep. 3, 2021, which claims the benefit of priority under 35 U.S.C. § 119(e) to U.S. Application No. 63/074,878 filed on Sep. 4, 2020. This document is incorporated by reference herein in its entirety.

### TECHNICAL FIELD

This disclosure generally relates to methods and compositions related to predicting cardiovascular disease (CVD) in an individual such as, for example, coronary heart disease (CHD).

### BACKGROUND

Cardiovascular disease (CVD), and particularly coronary heart disease (CHD), is the most common type of heart disease and was responsible for over 360,000 deaths in the United States in 2017. In order to decrease this toll, a number of risk estimators have been developed to better identify those with or at risk for CHD. Beginning with the Framingham Risk Score (FRS) and more recently, the ASCVD Pooled Cohort Equation (PCE), these tools capture variance in key physiological parameters, such as serum lipid levels, known to be associated with risk for CVD, including CHD.

Despite the magnitude of these efforts, current risk estimators often lack in sensitivity and specificity. As a result, there is a need for alternative stratification approaches for CVD.

### SUMMARY

Methods and compositions for predicting the incidence or risk of cardiovascular disease (CVD) are provided. For example, methods and compositions for predicting the one-year, three-year or five-year incidence of coronary heart disease (CHD) are described herein. The general principals apply to other windows of incidence (e.g., one-month, six-month, two-year, or ten-year) as well as the incidence or prevalence of other types of CVD including, without limitation, CHD, stroke, arrhythmia, cardiac arrest, and congestive heart failure. Specifically, methods and compositions for determining the methylation status of at least one CpG locus and at least one single nucleotide polymorphism (SNP) are described.

In one aspect, kits for determining methylation status of at least one CpG dinucleotide and a genotype of at least one single-nucleotide polymorphism (SNP) are provided. Such kits typically include at least one first nucleic acid primer at least 8 nucleotides in length that is complementary to a bisulfite-converted nucleic acid sequence comprising a first CpG dinucleotide at a GC locus selected from the group consisting of cg00300879, cg09552548, and cg14789911 or at a second CpG dinucleotide in linkage disequilibrium with the first CpG dinucleotide at a GC locus selected from the group consisting of cg00300879, cg09552548, and cg14789911, wherein the linkage disequilibrium has a value of R>0.3, wherein the at least one first nucleic acid primer detects a methylated or unmethylated CpG dinucleotide, and at least one second nucleic acid primer at least 8 nucleotides in length that is complementary to a DNA sequence or a bisulfite-converted DNA sequence of a first SNP selected from the group consisting of rs11716050, rs6560711, rs3735222, rs6820447, and rs9638144 or a second SNP in linkage disequilibrium with the first SNP selected from the group consisting of rs11716050, rs6560711, rs3735222, rs6820447, and rs9638144, wherein the linkage disequilibrium has a value of R>0.3.

In some embodiments, the at least one first nucleic acid primer detects the unmethylated CpG dinucleotide. In some embodiments, the at least one first nucleic acid primer detects the methylated CpG dinucleotide.

In some embodiments, the kits described herein further including at least a third nucleic acid primer at least 8 nucleotides in length that is complementary to a nucleic acid sequence upstream of the CpG dinucleotide. In some embodiments, the kits further include at least a third nucleic acid primer at least 8 nucleotides in length that is complementary to a nucleic acid sequence downstream of the CpG dinucleotide.

In some embodiments, the at least one first nucleic acid primer comprises one or more nucleotide analogs. In some embodiments, the at least one first nucleic acid primer comprises one or more synthetic or non-natural nucleotides.

In some embodiments, the kits described herein further include a solid substrate to which the at least one first nucleic acid primer is bound. In some embodiments, the substrate is a polymer, glass, semiconductor, paper, metal, gel or hydrogel. In some embodiments, the solid substrate is a microarray or microfluidics card.

In some embodiments, the kits described herein further include a detectable label.

In another aspect, methods of determining the presence of biomarkers associated with predicting CHD in a biological sample from a patient is provided. Such methods typically include (a) providing a first portion of the biological sample and a second portion of the biological sample, wherein the nucleic acid from at least the first portion is bisulfite converted; (b) contacting the first portion of the biological sample with a first oligonucleotide primer at least 8 nucleotides in length that is complementary to a sequence that comprises a first CpG dinucleotide at a GC locus selected from the group consisting of cg00300879, cg09552548, and cg14789911, or a second CpG dinucleotide in linkage disequilibrium with the first CpG dinucleotide at a GC locus selected from the group consisting of cg00300879, cg09552548, and cg14789911, wherein the linkage disequilibrium has a value of R>0.3, wherein the first nucleic acid primer detects a methylated or unmethylated CpG dinucleotide; and (c) contacting the second portion of the biological sample with a nucleic acid primer at least 8 nucleotides in length that is complementary to a DNA sequence or a bisulfite-converted DNA sequence of a first SNP selected from the group consisting of rs11716050, rs6560711, rs3735222, rs6820447, and rs9638144 or a second SNP in linkage disequilibrium with the first SNP selected from the group consisting of rs11716050, rs6560711, rs3735222, rs6820447, and rs9638144, wherein the linkage disequilibrium has a value of R>0.3. Generally, the percentage of methylation of the CpG dinucleotide at the GC locus selected from the group consisting of cg00300879, cg09552548, and cg14789911, and the identity of the nucleotide at the first SNP selected from the group consisting of rs11716050, rs6560711, rs3735222, rs6820447, and rs9638144 or the second SNP in linkage disequilibrium with the first SNP are biomarkers associated with the incidence of CHD.

In some embodiments, the biological sample is selected from the group consisting of blood and saliva.

In some embodiments, the at least one first nucleic acid primer detects the unmethylated CpG dinucleotide. In some embodiments, the at least one first nucleic acid primer detects the methylated CpG dinucleotide.

In some embodiments, the at least one first nucleic acid primer comprises one or more nucleotide analogs. In some embodiments, the at least one first nucleic acid primer comprises one or more synthetic or non-natural nucleotides.

In some embodiments, the window of incidence is three years.

In still a further aspect, methods of determining the presence of a biomarker associated with CHD in a patient sample are provided. Such methods typically include (a) isolating nucleic acid sample from the patient sample, (b) performing a genotyping assay on a first portion of the nucleic acid sample to detect the presence of at least one SNP, wherein the at least one SNP is a first SNP from Appendix C and/or is a second SNP in linkage disequilibrium (R>0.3) with a first SNP from Appendix C to obtain genotype data; and/or (c) bisulfite converting the nucleic acid in a second portion of the nucleic acid and performing methylation assessment on the second portion of the nucleic acid sample to detect methylation status of at least one CpG site from Appendix A and/or a CpG site collinear (R>0.3) with a CpG from Appendix A to obtain methylation data; and (d) inputing the genotype data from step (b) and/or methylation data from step (c) into an algorithm that accounts for at least one SNP main effect and/or at least one CpG main effect and/or at least one interaction effect, wherein the algorithm is a machine learning algorithm capable of accounting for linear and non-linear effects.

In some embodiments, the at least one interaction effect is selected from the group consisting of a gene-environment interaction (SNPxCpG) effect, a gene-gene interaction (SNPxSNP) effect, and an environment-environment interaction (CpGxCpG) effect. In some embodiments, the at least one interaction effect is a gene-environment interaction effect (SNPxCpG) between a CpG site from Appendix A or a CpG site that is collinear (R>0.3) with a CpG site from Appendix A and a SNP from Appendix C or a SNP within moderate linkage disequilibrium (R>0.3) from a SNP from Appendix C. In some embodiments, the at least one interaction effect is an environment-environment interaction effect (CpGxCpG) between at least two CpG sites from Appendix A.

In some embodiments, one or both of the at least two CpG sites are collinear (R>0.3) with one or both of the at least two CpG sites from Appendix A. In some embodiments, the at least one interaction effect is a gene-gene interaction effect (SNPxSNP) between at least two SNPs from Appendix C. In some embodiments, one or both of the at least two SNPs are collinear (R>0.3) with one or both of the at least two SNPs from Appendix C.

In some embodiments, the biological sample is a saliva sample.

In another aspect, systems for determining methylation status of at least one CpG dinucleotide and a genotype of at least one single-nucleotide polymorphism (SNP) are provided. Such systems typically include: a nucleic acid isolation module configured to isolate a nucleic acid sample from a subject sample; a genotyping assay module configured to perform a genotyping assay on a first portion of the nucleic acid sample to detect the presence of at least one SNP, wherein the at least one SNP is a first SNP from Appendix C and/or is a second SNP in linkage disequilibrium (R>0.3) with a first SNP from Appendix C to obtain genotype data; a methylation assay module configured to bisulfite convert the nucleic acid in a second portion of the nucleic acid and perform a methylation assessment on a second portion of the nucleic acid sample to detect methylation status of at least one CpG site from Appendix A and/or a CpG site collinear (R>0.3) with a CpG from Appendix A to obtain methylation data; and an identification system configured to account for at least one SNP main effect and/or at least one CpG main effect and/or at least one interaction effect based on the genotype data from step (b) and/or methylation data from step (c).

In some embodiments, such systems further include an output module configured to provide an output based on an identification by the identification system, wherein the identification accounts for at least one SNP main effect and/or at least one CpG main effect and/or at least one interaction effect based on the genotype data from step (b) and/or methylation data from step (c).

In some embodiments, the algorithm is a machine learning algorithm capable of accounting for linear and non-linear effects.

In yet another aspect, non-transitory computer-readable media storing instructions executable by a processing device to perform operations are provided. Such operations typically include accounting for at least one SNP main effect and/or at least one CpG main effect and/or at least one interaction effect based on genotype data and/or methylation data, wherein: (i) the genotype data is based on a genotyping assay on a first portion of a nucleic acid sample isolated from a subject sample to detect the presence of at least one SNP, wherein the at least one SNP is a first SNP from Appendix C and/or is a second SNP in linkage disequilibrium (R>0.3) with a first SNP from Appendix C to obtain the genotype data; and (ii) the methylation data is based on a methylation assay on a bisulfite converted nucleic acid in a second portion of the nucleic acid sample to detect methylation status of at least one CpG site from Appendix A and/or a CpG site collinear (R>0.3) with a CpG from Appendix A to obtain methylation data.

In some embodiments, the operations further include providing an output based on the accounting. Representative outputs, without limitation, include one or more of storing a report based on the accounting to another non-transitory computer-readable medium, modifying a display based on the accounting, triggering an audible alert based on the accounting, triggering a haptic or vibratory alert based on the accounting, triggering the printing of a report based on the accounting, or triggering the delivery of a therapeutic based on the accounting.

The integrated genetic-epigenetic model described herein provides several advantages and benefits. The first is the overall sensitivity across cohorts. On average, in the Intermountain Healthcare (IM) cohort, the typical risk calculators accurately identify 5 out of 10 individuals at high risk for an incident event compared to the integrated genetic-epigenetic model described herein, which accurately identifies 7 out of 10 individuals. The second is with respect to the performance of standard risk calculators by gender. On average, in the IM cohort, the typical risk calculators accurately identify 5 of 10 men and 4 of 10 women at risk for an incident event. On average, in the IM cohort, the integrated genetic-epigenetic tool described herein accurately identifies 7 of 10 men and 7 of 10 women at risk for an incident event. Thus, the integrated genetic-epigenetic model described herein does not exhibit gender gap in its ability to identify men and women at risk for an incident event.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limited to predicting incident CHD. All publications, patent applications, patents, and other references mentioned herein are incorporated by reference in their entirety.

### DESCRIPTION OF DRAWINGS

FIG. 1 is a graph showing the distribution of the number of incident cases over three years in the Framingham Heart Study Offspring cohort.

FIG. 2 is a graph showing the distribution of the number of incident cases over three years in the Intermountain Healthcare cohort.

FIG. 3 is a graph showing the ROC curves of the integrated genetic-epigenetic model for three-year incidence CHD risk assessment in the FHS training, FHS test, IM validation and IM test sets.

FIG. 4 is a graph showing the average AUC of the baseline integrated genetic-epigenetic model compared to models with only SNPs, only DNA methylation loci and the addition of conventional risk factors and Polygenic Risk Score.

FIG. 5 shows a Kaplan-Meier survival curve of the high and low risk groups.

FIG. 6 shows a Kaplan-Meier survival curve for high, intermediate and low prognostic scores.

FIG. 7 shows the correlation (r−0.94) between digital PCR and array DNA methylation values for cg00300879.

FIG. 8 is a block diagram of an example cardiovascular disease classification system.

FIG. 9 is a flow diagram of an example process for cardiovascular disease classification.

FIG. 10 is a block diagram of example computing devices.

FIGS. 11A-11C are graphs showing the relationship of change in increases in cg05575921 methylation seen in response to smoking cessation to changes in methylation at each of the three loci associated with cardiac risk between study entry and study exit (3 months) in the 20 subjects who had biochemically verified smoking cessation. FIG. 11A is a plot of the change of methylation status at cg14789911 with respect to the change of methylation status at cg05575921. FIG. 11A shows the relationship between increases in methylation at cg05575921 seen in response to smoking cessation and changes in methylation at cg14789911 between study entry to study exit (3 months) in the 20 subjects who had biochemically verified smoking cessation. A negative A indicates an increase in methylation at the marker associated with cardiac risk. FIG. 11B is a plot of the change of methylation status at cg09552548 with respect to the change of cg05575921 methylation. FIG. 11B shows the relationship between increases of cg05575921 methylation seen in response to smoking cessation and changes in methylation at cg09552548 between study entry to study exit (3 months) in the 20 subjects who had biochemically verified smoking cessation. A negative A indicates an increase in methylation at the marker associated with cardiac risk. FIG. 11C is a plot of the change of cg00300879 with respect to the change cg05575921 methylation. The change illustrated in FIG. 11C is significant after Bonferroni correction (Adj R2 0.26, p<0.04). FIG. 11C shows the relationship between increases of cg05575921 methylation seen in response to smoking cessation and changes in methylation at cg00300879 between study entry to study exit (3 months) in the 20 subjects who had biochemically verified smoking cessation. A negative A indicates an increase in methylation at the marker associated with cardiac risk.

### DETAILED DESCRIPTION

Recent risk prediction strategies have taken advantage of the rapid advancements in assessing genome-wide genetic or transcriptional variation. Though each of these approaches have had some success, their clinical impact has been limited. In particular, those relying only on genetic information have a clear ceiling in predictive capacity, are potentially sensitive to ethnic stratification, and, because genotype is static, cannot be used to monitor changes in disease status.

Recent advances in genome-wide epigenetic profiling techniques have raised the possibility that DNA methylation assessments of peripheral blood DNA may serve as a mechanism for more accurate prediction of cardiovascular disease or mortality. Prediction models that only account for epigenetic signatures, however, fail to account for confounding genetic variation, which affects the vast majority of the environmentally responsive methylome. This may result in models that lack robustness with respect to generalizability, especially in different ethnic groups.

As a result, we have developed a highly sensitive, clinically implementable integrated genetic-epigenetic risk assessment tool capable of identifying those at risk of cardiovascular disease (e.g., having a heart attack or sudden cardiac death) within one year, three years or five years. As shown herein, the methylation status of one or more particular CpG dinucleotides in combination with the genotype at one or more particular loci (e.g., CH3xSNP) can be used to predict the incidence (e.g., one-year, three-year, five-year) of cardiovascular disease (CVD) including coronary heart disease (CHD).

As described herein, biomarkers described herein can be used in the diagnosis and prognosis of cardiovascular diseases and events. The terms “marker” and “biomarker” can be used interchangeably. As used herein, a biomarker generally refers to a measurable or detectable biological moiety (e.g., the presence or amount of a protein, a genetic and/or histological component). As described in more detail below, the biomarkers used herein typically are associated with cardiovascular disease.

### DNA Methylation

DNA does not exist as naked molecules in the cell. For example, DNA is associated with proteins called histones to form a complex substance known as chromatin. Chemical modifications of the DNA or the histones alter the structure of the chromatin without changing the nucleotide sequence of the DNA. Such modifications are described as “epigenetic” modifications of the DNA. Changes to the structure of the chromatin can have a profound influence on gene expression. If the chromatin is condensed, factors involved in gene expression may not have access to the DNA, and the genes will be switched off. Conversely, if the chromatin is “open,” the genes can be switched on. Some important forms of epigenetic modification are DNA methylation and histone deacetylation.

DNA methylation is a chemical modification of the DNA molecule itself and is carried out by an enzyme called DNA methyltransferase. Methylation can directly switch off gene expression by preventing transcription factors binding to promoters. A more general effect is the attraction of methyl-binding domain (MBD) proteins. These are associated with further enzymes called histone deacetylases (HDACs), which function to chemically modify histones and change chromatin structure. Chromatin-containing acetylated histones are open and accessible to transcription factors, and the genes are potentially active. Histone deacetylation causes the condensation of chromatin, making it inaccessible to transcription factors and causing the silencing of genes.

CpG islands are short stretches of DNA in which the frequency of the CpG sequence is higher than other regions. The “p” in the term CpG indicates that cysteine (“C”) and guanine (“G”) are connected by a phosphodiester bond. CpG islands are often located around promoters of housekeeping genes and many regulated genes. At these locations, the CG sequence is not methylated. By contrast, the CG sequences in inactive genes are usually methylated to suppress their expression.

As used herein, the term “methylation status” means the determination whether a certain target DNA, such as a CpG dinucleotide, is methylated or is unmethylated. As used herein, the term “CpG dinucleotide repeat motif” means a series of two or more CpG dinucleotides positioned in a DNA sequence.

About 56% of human genes and 47% of mouse genes are associated with CpG islands. Often, CpG islands overlap the promoter and extend about 1000 base pairs downstream into the transcription unit. Identification of potential CpG islands during sequence analysis helps to define the extreme 5′ ends of genes, something that is notoriously difficult with cDNA-based approaches. The methylation of a CpG island can be determined by a skilled artisan using any method suitable to determine such methylation. For example, the skilled artisan can use a bisulfite reaction-based method for determining such methylation.

The present disclosure provides methods to determine the nucleic acid methylation of one or more loci in a subject in order to predict the three-year clinical course and eventual outcome of subjects having CVD.

Genetic screening (also called genotyping or molecular screening) can be broadly defined as testing to determine if a subject has a genetic marker that either causes a disease state or is “linked” to the genetic component causing the disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in “linkage disequilibrium” with one another because, in a given population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

It would be understood that linkage disequilibrium can be quantitated (using, for example, the Pearson correlation (R) or co-inheritance of alleles (D′)). For example, a low level of linkage can be reflected in a correlation (e.g., R value) of about 0.1 or less, a moderate level of linkage is reflected in a R value of about 0.3, while a high level of linkage is reflected in a R value of 0.5 or greater. It also would be understood that, when referring to methylation (i.e. CpGs), collinearity (with an R value) is used as a determination of the linear strength of the association between two CpGs (e.g., a low level of collinearity can be reflected by an R value of about 0.1 or less; a moderate level of collinearity can be reflected by an R value of about 0.3; and a high level of collinearity can be reflected by an R value of about 0.5 or greater).

In particular, in certain embodiments of the disclosure, the methods may be practiced as follows. A sample, such as a blood sample, is taken from a subject. In certain embodiments, a single cell type, e.g., lymphocytes, basophils, or monocytes isolated from the blood, may be isolated for further testing. The DNA is harvested from the sample and examined to determine the methylation of one or more loci. For example, the DNA of interest can be treated with bisulfite to deaminate unmethylated cytosine residues to uracil. Since uracil base pairs with adenosine, thymidines are incorporated into subsequent DNA strands in the place of unmethylated cytosine residues during subsequence PCR amplifications. Next, the target sequence is amplified by PCR, and probed with a loci-specific probe. Depending on the particular sequence of the probe used, only the methylated or unmethylated DNA will bind to the probe.

Methods of determining the subject nucleic acid profile are well known to a skilled artisan and include any of the well-known detection methods. Various PCR methods are described, for example, in PCR Primer: A Laboratory Manual, Dieffenbach 7 Dveksler, Eds., Cold Spring Harbor Laboratory Press, 1995. Other methods include, but are not limited to, nucleic acid quantification, restriction enzyme digestion, DNA sequencing, hybridization technologies, such as Southern Blotting, amplification methods such as Ligase Chain Reaction (LCR), Nucleic Acid Sequence Based Amplification (NASBA), Self-sustained Sequence Replication (SSR or 3SR), Strand Displacement Amplification (SDA), and Transcription Mediated Amplification (TMA), Quantitative PCR (qPCR), or other DNA analyses, as well as RT-PCR, in vitro translation, Northern blotting, and other RNA analyses. In another embodiment, hybridization on a microarray is used.

### Single Nucleotide Polymorphism (SNP) Genotyping

Traditional methods for the screening of heritable diseases have depended on either the identification of abnormal gene products (e.g., sickle cell anemia) or an abnormal phenotype (e.g., mental retardation). With the development of simple and inexpensive genetic screening methodology, it is now possible to identify polymorphisms that indicate a propensity to develop disease, even when the disease is of polygenic origin.

Single nucleotide polymorphism (SNP) genotyping measures genetic variations of SNPs between members of a species. A SNP is a single base pair change at a specific locus, usually consisting of two alleles (where the rare allele frequency is >1%). SNPs are very common. Because SNPs are conserved during evolution, they have been proposed as markers for use in quantitative trait loci (QTL) analysis and in association studies in place of microsatellites. Many different SNP genotyping methods are known, including hybridization-based methods (such as Dynamic allele-specific hybridization, molecular beacons, and SNP microarrays) enzyme-based methods (including restriction fragment length polymorphism, PCR-based methods, flap endonuclease, primer extension, 5′-nuclease, and oligonucleotide ligation assay), other post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex and surveyor nuclease assay), and sequencing (such as “next generation” sequencing). See, e.g., U.S. Pat. No. 7,972,779.

A plurality of alleles at a locus can arise from one or more polymorphisms in a region of a gene that encodes a polypeptide or in a regulatory control sequence that affects expression of the polypeptide, such as a promoter or polyadenylation sequence. Alternatively, alleles can arise from one or more polymorphisms at a locus distal to a gene that encodes a polypeptide or in a regulatory control sequence. A polymorphism can affect a polypeptide at a transcriptional or a translational level (e.g., a polypeptide's transcription rate, translation rate, degradation rate, and/or activity). Allelic differences can be characterized in a sample from a single subject or from a plurality of subjects using methods that are known to a skilled artisan. Such methods can include, but are not limited to, measuring the potential for a polynucleotide sequence to be expressed and/or measuring an amount of an encoded polypeptide. Methods are available that can detect proteins or nucleic acids directly or indirectly, and assay methods are specifically contemplated to include screening for the presence of particular sequences or structures of nucleic acids or polypeptides using, e.g., any of various known microarray technologies.

It will be fully appreciated by the skilled artisan that the allele need not have previously been shown to have had any link or association with the disorder phenotype. Instead, an allele and a pathogenic environmental risk factor can interact to predict a predisposition to a disorder phenotype even when neither the allele nor the risk factor bears any direct relation to the disorder phenotype.

Genetic screening (also called genotyping or molecular screening) can be broadly defined as testing to determine if a subject has mutations (or alleles or polymorphisms) that either cause a disease state or are “linked” to the mutation causing a disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in “linkage disequilibrium” with one another because, in a given population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

It would be understood that linkage disequilibrium can be quantitated (using, for example, the Pearson correlation (R) or co-inheritance of alleles (D′)). For example, a low level of linkage can be reflected in a correlation (e.g., R value) of about 0.1 or less, a moderate level of linkage is reflected in a R value of about 0.3, while a high level of linkage is reflected in a R value of 0.5 or greater. It also would be understood that, when referring to methylation (i.e. SNPs), collinearity (with an R value) is used as a determination of the linear strength of the association between two SNPs (e.g., a low level of collinearity can be reflected by an R value of about 0.1 or less; a moderate level of collinearity can be reflected by an R value of about 0.3; and a high level of collinearity can be reflected by an R value of about 0.5 or greater).

While the frequency of meiotic recombination between two markers is generally proportional to the physical distance between them on the chromosome, the occurrence of “hot spots” as well as regions of repressed chromosomal recombination can result in discrepancies between the physical and recombinational distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore, identification of a haplotype that spans or is linked to a disease-causing mutational change serves as a predictive measure of an individual's likelihood of having inherited that disease- causing mutation. Such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases.

The statistical correlation between a disorder and a polymorphism does not necessarily indicate that the polymorphism directly causes the disorder. Rather the correlated polymorphism may be a benign allelic variant which is linked to (i.e., in linkage disequilibrium with) a disorder-causing mutation that has occurred in the recent evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus, these other polymorphic loci will also be prognostic or diagnostic of the likelihood of having inherited the disease-causing polymorphic locus. A broad-spanning haplotype (describing the typical pattern of co-inheritance of alleles of a set of linked polymorphic markers) can be targeted for diagnostic purposes once an association has been drawn between a particular disease or condition and a corresponding haplotype. Thus, the determination of an individual's likelihood for developing a particular disease of condition can be made by characterizing one or more disease-associated polymorphic alleles (or even one or more disease-associated haplotypes) without necessarily determining or characterizing the causative genetic variation.

Many methods are available for detecting specific alleles at polymorphic loci. Certain methods for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the genome is estimated to be on average 1 per 1,000 base pairs. SNPs are most frequently bi-allelic, or occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

In one embodiment, allelic profiling can be accomplished using a nucleic acid microarray. The genetic testing field is rapidly evolving and, as such, the skilled artisan will appreciate that a wide range of profiling tests exist, and will be developed, to determine the allelic profile of individuals in accord with the disclosure.

### Nucleic Acids and Polypeptides

The term “nucleic acid” refers to deoxyribonucleotides or ribonucleotides and polymers thereof in either single-or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The terms “nucleic acid,” “nucleic acid molecule,” or “polynucleotide” are used interchangeably and may also be used interchangeably with gene, cDNA, DNA and/or RNA encoded by a gene.

The term “nucleotide sequence” refers to a polymer of DNA or RNA which can be single-stranded or double-stranded, optionally containing synthetic, non-natural or altered nucleotide bases capable of incorporation into DNA or RNA polymers. A DNA molecule or polynucleotide is a polymer of deoxyribonucleotides (A, G, C, and T), and an RNA molecule or polynucleotide is a polymer of ribonucleotides (A, G, C and U).

A “gene,” for the purposes of the present disclosure, includes a DNA region encoding a gene product, as well as all DNA regions, which regulate the production of the gene product, whether or not such regulatory sequences are adjacent to coding and/or transcribed sequences. The term “gene” is used broadly to refer to any segment of nucleic acid associated with a biological function. Genes include coding sequences and/or the regulatory sequences required for their expression. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. For example, “gene” refers to a nucleic acid fragment that expresses mRNA, functional RNA, or specific protein, including regulatory sequences. “Functional RNA” refers to sense RNA, antisense RNA, ribozyme RNA, siRNA, or other RNA that may not be translated but yet has an effect on at least one cellular process. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

“Gene expression” refers to the conversion of the information, contained in a gene, into a gene product. It refers to the transcription and/or translation of an endogenous gene, heterologous gene or nucleic acid segment, or a transgene in cells. In addition, expression refers to the transcription and stable accumulation of sense (mRNA) or functional RNA. Expression may also refer to the production of protein. The term “altered level of expression” refers to the level of expression in transgenic cells or organisms that differs from that of normal or untransformed cells or organisms.

A gene product can be the transcriptional product of a gene (e.g., mRNA, tRNA, rRNA, antisense RNA, ribozyme, structural RNA or any other type of RNA) or a protein produced by translation of an mRNA. Gene products also include RNAs that are modified, by processes such as capping, polyadenylation, methylation, and editing, and proteins modified by, for example, methylation, acetylation, phosphorylation, ubiquitination, ADP-ribosylation, myristilation, and glycosylation. The term “RNA transcript” refers to the product resulting from RNA polymerase-catalyzed transcription of a DNA sequence. When the RNA transcript is a complementary copy of the DNA sequence, it is referred to as the primary transcript; a RNA sequence derived from post-transcriptional processing of the primary transcript is referred to as the mature RNA. “Messenger RNA” (mRNA) refers to the RNA that lacks introns and that can be translated into protein by the cell. “cDNA” refers to a single-or a double-stranded DNA that is complementary to and derived from mRNA. “Functional RNA” refers to sense RNA, antisense RNA, ribozyme RNA, siRNA, or other RNA that may not be translated but yet has an effect on at least one cellular process.

A “coding sequence” or a sequence that “encodes” a polypeptide is a nucleic acid molecule that is transcribed (in the case of DNA) and/or translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral (e.g., DNA viruses and retroviruses) or prokaryotic DNA, and synthetic DNA sequences. A transcription termination sequence can be located 3′ to the coding sequence.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences.

Certain embodiments of the disclosure encompass isolated or substantially purified nucleic acid compositions. In the context of the present disclosure, an “isolated” or “purified” DNA molecule or RNA molecule is a DNA molecule or RNA molecule that exists apart from its native environment and is, therefore, not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

By “fragment” is intended a polypeptide consisting of only a part of the intact full-length polypeptide sequence and structure. The fragment can include a C-terminal deletion, an N-terminal deletion, and/or an internal deletion of the native polypeptide. A fragment of a protein will generally include at least about 5-100 contiguous amino acid residues of the full-length molecule (e.g., at least about 15-25 contiguous amino acid residues of the full-length molecule, at least about 20-50 or more contiguous amino acid residues of the full-length molecule, or any integer between 5 amino acids and the full-length sequence).

“Naturally occurring” is used to describe a composition that can be found in nature as distinct from being artificially produced. For example, a nucleotide sequence present in an organism, which can be isolated from a source in nature and which has not been intentionally modified by a person in the laboratory, is naturally occurring.

A “5′ non-coding sequence” refers to a nucleotide sequence located 5′ (upstream) to the coding sequence. 5′ non-coding sequences are present in the fully processed mRNA upstream of the initiation codon and may affect processing of the primary transcript to mRNA, mRNA stability or translation efficiency. A “3′ non-coding sequence” refers to nucleotide sequences located 3′ (downstream) to a coding sequence and may include polyadenylation signal sequences and other sequences encoding regulatory signals capable of affecting mRNA processing or gene expression.

A “promoter” refers to a nucleotide sequence, usually upstream (5′) to its coding sequence, which directs and/or controls the expression of the coding sequence by providing the recognition for RNA polymerase and other factors required for proper transcription. “Promoter” can include a minimal promoter that is a short DNA sequence comprised of a TATA-box and other sequences that serve to specify the site of transcription initiation, to which regulatory elements are added for control of expression. “Promoter” also can refer to a nucleotide sequence that includes a minimal promoter plus one or more regulatory elements (e.g., enhancers) that are capable of controlling the expression of a coding sequence or functional RNA. Promoters may be derived in their entirety from a native sequence, or be composed of different elements derived from different promoters found in nature, or even be comprised of synthetic DNA sequences. A promoter may also contain DNA sequences that are involved in the binding of protein factors that control the effectiveness of transcription initiation in response to physiological or developmental conditions. “Constitutive expression” refers to expression using a constitutive promoter. “Conditional” and “regulated expression” refer to expression controlled by a regulated promoter.

An “enhancer” is a DNA sequence that can stimulate promoter activity. An enhancer may be an innate element of the promoter or a heterologous element inserted to enhance the level or tissue specificity of a promoter. Enhancers often are capable of operating in both orientations, and are capable of functioning even when moved either upstream or downstream from the promoter. Both enhancers and other regulatory elements within a promoter bind sequence-specific DNA-binding proteins that mediate their effects.

“Operably-linked” refers to the association of nucleic acid sequences on a single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. In addition, expression refers to the transcription and stable accumulation of sense (mRNA) or functional RNA. Expression may also refer to the production of protein. The term “altered level of expression” refers to a level of expression in cells or organisms that differs from that of normal cells or organisms.

For sequence comparison, typically one sequence acts as a reference sequence to which test sequences are compared. When using a sequence comparison algorithm, test and reference sequences are input into a computer, and sequence algorithm program parameters are designated. The sequence comparison algorithm then calculates the percent sequence identity for the test sequence(s) relative to the reference sequence, based on the designated algorithm parameters.

The following terms are used to describe the sequence relationships between two or more nucleic acids or polynucleotides: (a) “reference sequence,” (b) “comparison window,” (c) “sequence identity,” (d) “percentage of sequence identity,” and (e) “as is for sequence comparison. A reference sequence may be a subset or the substantial identity.” As used herein, “reference sequence” is a defined sequence used as a b entirety of a specified sequence; for example, as a segment of a full-length cDNA or gene sequence, or the complete cDNA or gene sequence. As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that, to avoid a high similarity to a reference sequence due to inclusion of gaps in the polynucleotide sequence, a gap penalty is typically introduced and is subtracted from the number of matches.

Methods of alignment of sequences for comparison are well-known in the art. Thus, the determination of percent identity between any two sequences can be accomplished using a mathematical algorithm. Non-limiting examples of such mathematical algorithms are the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, JMB, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993)).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., J. Mol. Biol., 215, 403 (1990)) are based on the algorithm of Karlin and Altschul, supra.

Software for performing BLAST analyses is publicly available through the National Center for Biotechnology Information. This algorithm involves first identifying high scoring sequence pairs (HSPs) by identifying short words of length “W” in the query sequence, which either match or satisfy some positive-valued threshold score T when aligned with a word of the same length in a database sequence. “T” is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0), and for amino acid sequences, a scoring matrix is used to calculate the cumulative score. Extension of the word hits in each direction are halted when the cumulative alignment score falls off by the quantity “X” from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

For purposes of the present disclosure, comparison of nucleotide sequences for determination of percent sequence identity to the promoter sequences disclosed herein may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and, therefore, do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percent sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, 99% or 100% sequence identity, compared to a reference sequence using one of the alignment programs described herein using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70% (e.g., 71%, 72%, 73%, 74%, 75%, 76%, 77%, 78%, 79%), at least 80% (e.g., 81%, 82%, 83%, 84%, 85%, 86%, 87%, 88%, 89%), at least 90% (e.g., 91%, 92%, 93%, or 94%), or even at least 95% (e.g., 96%, 97%, 98%, 99%, or 100%).

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, J. Mol. Biol., 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the disclosure also provides nucleic acid molecules and peptides that are substantially identical to the nucleic acid molecules and peptides presented herein.

Another indication that nucleotide sequences are substantially identical is if two molecules hybridize to each other under stringent conditions. Hybridization of nucleic acids is discussed in more detail below.

### Oligonucleotide Primers and Probes

As used herein, “primer,” “probe,” and “oligonucleotide” are used interchangeably. The term “nucleic acid probe” or a “probe specific for” a nucleic acid refers to a nucleic acid sequence that has at least about 80%, e.g., at least about 90%, e.g., at least about 95% contiguous sequence identity or homology to the nucleic acid sequence encoding the targeted sequence of interest. A probe (or oligonucleotide or primer) of the disclosure is at least about 8 nucleotides in length (e.g., at least about 8-50 nucleotides in length, e.g., at least about 10-40, e.g., at least about 15-35 nucleotides in length). The oligonucleotide probes or primers of the disclosure may comprise at least about eight nucleotides at the 3′ of the oligonucleotide that have at least about 80%, e.g., at least about 85%, e.g., at least about 90%, e.g., at least about 95% contiguous identity to the targeted sequence of interest. Primer pairs are useful for determination of the nucleotide sequence of a particular SNP using PCR. The pairs of single-stranded DNA primers can be annealed to sequences within or surrounding the SNP in order to prime amplifying DNA synthesis of the SNP itself. The first step of the process involves contacting a biological sample obtained from a subject, which sample contains nucleic acid, with at least one primer to form a hybridized DNA. The oligonucleotide primers that are useful in the methods of the present disclosure can be any primer comprised of about 8 bases up to about 80 or 100 bases or more. In one embodiment of the present disclosure, the primers are between about 10 and about 20 bases.

The primers themselves can be synthesized using techniques that are well known in the art. Generally, the primers can be made using oligonucleotide synthesizing machines that are commercially available.

The primers or probes of the present disclosure can be labeled using techniques known to those of skill in the art. For example, the labels used in the assays of disclosure can be primary labels (where the label comprises an element that is detected directly) or secondary labels (where the detected label binds to a primary label, e.g., as is common in immunological labeling). An introduction to labels (also called “tags”), tagging or labeling procedures, and detection of labels is found in Polak and Van Noorden (1997) Introduction to Immunocytochemistry, second edition, Springer Verlag, N.Y. and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements. Useful primary and secondary labels in the present disclosure can include spectral labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™, and the like), radiolabels (e.g., ³H, ²⁵I, ³⁵S, ¹⁴C, ³²P, ³³P), enzymes (e.g., horse-radish peroxidase, alkaline phosphatase) spectral colorimetric labels such as colloidal gold or colored glass or plastic (e.g., polystyrene, polypropylene, latex) beads. The label may be coupled directly or indirectly to a component of the detection assay (e.g., the labeled nucleic acid) according to methods well known in the art. As indicated above, a wide variety of labels may be used, with the choice of label depending on sensitivity required, ease of conjugation with the compound, stability requirements, available instrumentation, and disposal provisions.

In general, a detector that monitors a probe-substrate nucleic acid hybridization is adapted to the particular label that is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeled nucleic acids is digitized for subsequent computer analysis.

Labels include those that use (1) chemiluminescence (using Horseradish Peroxidase and/or Alkaline Phosphatase with substrates that produce photons as breakdown products) with kits being available, e.g., from Molecular Probes, Amersham, Boehringer-Mannheim, and Life Technologies/Gibco BRL; (2) color production (using both Horseradish Peroxidase and/or Alkaline Phosphatase with substrates that produce a colored precipitate) (kits available from Life Technologies/Gibco BRL, and Boehringer-Mannheim); (3) hemifluorescence using, e.g., Alkaline Phosphatase and the substrate AttoPhos (Amersham) or other substrates that produce fluorescent products, (4) fluorescence (e.g., using Cy-5 (Amersham), fluorescein, and other fluorescent labels); (5) radioactivity using kinase enzymes or other end-labeling approaches, nick translation, random priming, or PCR to incorporate radioactive molecules into the labeled nucleic acid. Other methods for labeling and detection will be readily apparent to one skilled in the art.

Fluorescent labels can be used and have the advantage of requiring fewer precautions in handling, and being amendable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Fluorescent moieties, which can be incorporated into a label, generally are known including Texas red, dixogenin, biotin, 1-and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes, flavin and many others. Many fluorescent labels are commercially available from the SIGMA Chemical Company (Saint Louis, MO), Molecular Probes, R&D systems (Minneapolis, MN), Pharmacia LKB Biotechnology (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersberg, MD), Fluka ChemicaBiochemika Analytika (Fluka Chemie AG, Buchs, Switzerland), and Applied Biosystems™ (Foster City, CA), as well as many other commercial sources known to one of skill.

Means of detecting and quantifying labels are well known to those of skill in the art. Thus, for example, when the label is a radioactive label, means for detection include a scintillation counter or photographic film as in autoradiography; and when the label is optically detectable, typical detectors include microscopes, cameras, phototubes, photodiodes and many other detection systems that are widely available.

Oligonucleotide primers or probes may be prepared having any of a wide variety of base sequences according to techniques that are well known in the art. Suitable bases for preparing an oligonucleotide primer or probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyluridine, dihydrouridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylamninomethyluridine, 5-methoxyaminomethyl-2-thiouridine, β,D-mannosylqueosine, 5-methloxycarbonylmethyluridine, 5-methoxyuridine, 2-methyltio-N6-isopentenyladenosine, N-((9-β-D-ribofuranosyl-2-methylthiopurine-6-yl) carbamoyl)threonine, N-((9-β-D-ribofuranosylpurine-6-yl) N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-O-methyl-5-methyluridine, 2′-O-methylurdine, wybutosine, and 3-(3-amino-3-carboxypropyl) uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the internucleotide bridging phosphate residues are modified phosphates, such as methyl phosphonates, methyl phosphonotlioates, phosphoroinorpholidates, phosphoropiperazidates and phosplioramidates (for example, every other one of the internucleotide bridging phosphate residues may be modified as described). The oligonucleotide may be a “peptide nucleic acid” such as described in Nielsen et al., Science, 254:1497-1500 (1991).

As used herein, a “single base pair extension probe” is a nucleic acid that selectively recognizes a single nucleotide polymorphism (i.e., either the A or the G of an A/G polymorphism). Generally, these probes take the form of a DNA primer (e.g., as in PCR primers) that are modified so that incorporation of the primer releases a fluorophore. One example of this is a TaqmanX probe that uses the 5′ exonuclease activity of the enzyme Taq Polymerase for measuring the amount of target sequences in the samples. TaqMan® probes consist of a 18-22 bp oligonucleotide probe, which is labeled with a reporter fluorophore at the 5′ end, and a quencher fluorophore at the 3′ end. Incorporation of the probe molecule into a PCR chain (which occurs because the probe set is contained in a mixture of PCR primers) liberates the reporter fluorophore from the effects of the quencher. The primer must be able to recognize the target binding site. Some primer extension probes can be “activated” directly by DNA polymerase without a full PCR extension cycle.

The only requirement is that the oligonucleotide probe should possess a sequence at least a portion of which is capable of binding to a known portion of the sequence of the DNA sample. The nucleic acid probes provided by the present disclosure are useful for a number of purposes.

### Methods of Detecting Nucleic Acids

### A. Amplification

According to the methods of the present disclosure, the amplification of DNA present in a biological sample may be carried out by any means known to the art. Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3SR”), the Qbeta replicase system, nucleic acid sequence-based amplification (or “NASBA”), the repair chain reaction (or “RCR”), and boomerang DNA amplification (or “BDA”).

The bases incorporated into the amplification product can be natural or modified bases (modified before or after amplification), and the bases can be selected to optimize subsequent electrochemical detection steps.

Polymerase chain reaction (PCR) can be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding, to the reaction product, an oligonucleotide probe capable of hybridizing to the reaction product (e.g., an oligonucleotide primer or probe of the present disclosure), the probe carrying a detectable label, and then detecting the label in accordance with known techniques. Various labels that can be incorporated into or operably linked to nucleic acids are well known in the art, such as radioactive, enzymatic, and florescent labels. Where the nucleic acid to be amplified is RNA, amplification may be carried out by initial conversion to DNA by reverse transcriptase in accordance with known techniques.

Strand displacement amplification (SDA) can be carried out in accordance with known techniques. For example, SDA can be carried out with a single amplification primer or a pair of amplification primers, with exponential amplification being achieved with the latter. In general, SDA amplification primers comprise, in the 5′ to 3′ direction, a flanking sequence (the DNA sequence of which is noncritical), a restriction site for the restriction enzyme employed in the reaction, and an oligonucleotide sequence (e.g., an oligonucleotide primer or probe as described herein) that hybridizes to the target sequence to be amplified and/or detected. The flanking sequence, which serves to facilitate binding of the restriction enzyme to the recognition site and provides a DNA polymerase priming site after the restriction site has been nicked, can be about 15 to 20 nucleotides in length. The restriction site is functional in the SDA reaction. For example, the oligonucleotide primer or probe portion can be about 13 to 15 nucleotides in length.

Ligase chain reaction (LCR) also can be carried out in accordance with known techniques. In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection then can be carried out in like manner as described above with respect to PCR.

According to the methods described herein, a particular SNP at a particular locus can be detected. Techniques that are useful in the methods described herein include, but are not limited to, direct DNA sequencing, PFGE analysis, allele-specific oligonucleotide (ASO), dot blot analysis and denaturing gradient gel electrophoresis, and are well known to a skilled artisan.

There are several methods that can be used to detect DNA sequence variation. Direct DNA sequencing, either manual sequencing or automated fluorescent sequencing can detect sequence variation. Another approach is the single-stranded conformation polymorphism assay (SSCA). This method does not detect all sequence changes, especially if the DNA fragment size is greater than 200 bp, but can be optimized to detect most DNA sequence variation. The reduced detection sensitivity is a disadvantage, but the increased throughput possible with SSCA makes it an attractive, viable alternative to direct sequencing for mutation detection on a research basis. The fragments that have shifted mobility on SSCA gels then can be sequenced to determine the exact nature of the DNA sequence variation. Other approaches based on the detection of mismatches between the two complementary DNA strands include clamped denaturing gel electrophoresis (CDGE), heteroduplex analysis (HA) and chemical mismatch cleavage (CMC). Once a sequence change has been identified, an allele specific detection approach such as allele specific oligonucleotide (ASO) hybridization can be utilized to rapidly screen large numbers of other samples for that same sequence change (e.g., mutation, polymorphism). Such a technique can utilize probes that are labeled with gold nanoparticles to yield a visual color result.

Detection of SNPs can be accomplished by sequencing the desired target region using techniques well known in the art. Alternatively, sequences can be amplified directly from a genomic DNA preparation from subject tissue using known techniques. The DNA sequence of the amplified sequences then can be determined.

There are several well known methods for a more complete, yet still indirect, test for confirming the presence of a mutant allele: 1) single stranded conformation analysis (SSCA); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and/or 6) allele-specific PCR. For allele-specific PCR, primers are used that hybridize at their 3′ ends to a particular allele. If the particular mutation is not present, an amplification product is not observed. Amplification Refractory Mutation System (ARMS) can also be used. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Other techniques for detecting insertions and deletions as known in the art can be used.

In the first three methods (SSCA, DGGE and RNase protection assay), a new electrophoretic band appears. SSCA detects a band that migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present disclosure, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of samples. An example of a mismatch cleavage technique is the RNase protection method. The riboprobe and either mRNA or DNA isolated from the tumor tissue are annealed (hybridized) together and subsequently digested with the enzyme RNase A that is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the mRNA or gene but can be a segment of either. If the riboprobe includes only a segment of the mRNA or gene, it will be desirable to use a number of these probes to screen the whole mRNA sequence for mismatches.

In similar fashion, DNA probes can be used to detect mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA that might contain a mutation can be amplified using PCR before hybridization.

### B. Hybridization

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a primer or probe nucleic acid and a target nucleic acid and embraces minor mismatches that can be accommodated by reducing the stringency of the hybridization media to achieve the desired detection of the target nucleic acid sequence.

Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

“Stringent conditions” are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5× SSC (0.75 M NaCl, 0.075 M sodium citrate), 50 mM sodium phosphate (pH 6.8), 0.1% sodium pyrophosphate, 5x Denhardt's solution, sonicated salmon sperm DNA (50 μg/ml), 0.1% SDS, and 10% dextran sulfate at 42° C., with washes at 42° C. in 0.2× SSC and 0.1% SDS. Other examples of stringent conditions are well known in the art.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched primer or probe sequence. Specificity is typically the function of post-hybridization washes, the critical factors being the ionic strength and temperature of the final wash solution. For DNA-DNA hybrids, the Tm can be approximated from the equation of Meinkoth and Wahl (1984); Tₘ 81.5° C.+16.6 (log M)+0.41 (% GC)−0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. Tm is reduced by about 1° C. for each 1% of mismatching; thus, Tm, hybridization, and/or wash conditions can be adjusted to hybridize to sequences of the desired identity. For example, if sequences with >90% identity are sought, the Tm can be decreased 10° C. Generally, stringent conditions are selected to be about 5° C. lower than the Tm for the specific sequence and its complement at a defined ionic strength and pH. However, severely stringent conditions can utilize a hybridization and/or wash at 1, 2, 3, or 4° C. lower than the Tm; moderately stringent conditions can utilize a hybridization and/or wash at 6, 7, 8, 9, or 10° C. lower than the Tm; low stringency conditions can utilize a hybridization and/or wash at 11, 12, 13, 14, 15, or 20° C. lower than the Tm. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), the SSC concentration can be increased so that a higher temperature can be used. Generally, highly stringent hybridization and wash conditions are selected to be about 5° C. lower than the Tm for the specific sequence at a defined ionic strength and pH.

An example of highly stringent wash conditions is 0.15 M NaCl at 72° C. for about 15 minutes. An example of stringent wash conditions is a 0.2× SSC wash at 65° C. for 15 minutes. Often, a high stringency wash is preceded by a low stringency wash to remove background signal. An example of a medium stringency wash for a duplex of, e.g., more than 100 nucleotides, is 1× SSC at 45° C. for 15 minutes. For short nucleotide sequences (e.g., about 10 to 50 nucleotides), stringent conditions typically involve salt concentrations of less than about 1.5 M, less than about 0.01 to 1.0 M, Na ion concentration (or other salts) at pH 7.0 to 8.3, and the temperature is typically at least about 30° C. and at least about 60° C. for long oligonucleotides (e.g., >50 nucleotides). Stringent conditions also can be achieved by the addition of destabilizing agents such as formamide. In general, a signal to noise ratio of 2× (or higher) than that observed for an unrelated oligonucleotide in the particular hybridization assay indicates detection of a specific hybridization. Nucleic acids that do not hybridize to each other under stringent conditions are still substantially identical if the proteins that they encode are substantially identical. This can occur, e.g., when a copy of a nucleic acid is created using the maximum codon degeneracy permitted by the genetic code.

Very stringent conditions can be equal to the Tₘ for a particular oligonucleotide. An example of stringent conditions for hybridization of complementary nucleic acids that have more than 100 complementary residues on a filter in a Southern or Northern blot is 50% formamide, e.g., hybridization in 50% formamide, 1 M NaCl, 1% SDS at 37° C., and a wash in 0.1× SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1× SSC at 55 to 60° C.

“Northern analysis” or “Northern blotting” is a method used to identify RNA sequences that hybridize to a known probe such as an oligonucleotide, DNA fragment, cDNA or fragment thereof, or RNA fragment. The probe can be labeled with a radioisotope such as 32P, by biotinylation or with an enzyme. The RNA to be analyzed can be usually electrophoretically separated on an agarose or polyacrylamide gel, transferred to nitrocellulose, nylon, or other suitable membrane, and hybridized with the probe, using standard techniques well known in the art.

Nucleic acid sample may be contacted with an oligonucleotide in any suitable manner known to those skilled in the art. For example, the DNA sample may be solubilized in solution, and contacted with the oligonucleotide by solubilizing the oligonucleotide in solution with the DNA sample under conditions that permit hybridization. Suitable conditions are well known to those skilled in the art. Alternatively, the DNA sample may be solubilized in solution with the oligonucleotide immobilized on a solid support, whereby the DNA sample may be contacted with the oligonucleotide by immersing the solid support having the oligonucleotide immobilized thereon in the solution containing the DNA sample.

The term “substrate” refers to any solid support to which an oligonucleotide may be attached. The substrate material may be modified, covalently or otherwise, with coatings or functional groups to facilitate binding of oligonucleotides. Suitable substrate materials include polymers, glasses, semiconductors, papers, metals, gels and hydrogels among others. Substrates may have any physical shape or size, e.g., plates, strips, or microparticles. The term “spot” refers to a distinct location on a substrate to which oligonucleotides of known sequence are attached. A spot may be an area on a planar substrate, or it may be, for example, a microparticle distinguishable from other microparticles. The term “bound” means affixed to the solid substrate. A spot is “bound” to the solid substrate when it is affixed in a particular location on the substrate for purposes of the screening assay.

In certain embodiments of the present disclosure, the substrate is a polymer, glass, semiconductor, paper, metal, gel or hydrogel. In certain embodiments of the present disclosure, a kit can further include a solid substrate and at least one control oligonucleotide, wherein the at least one control oligonucleotide is bound onto the substrate in a distinct spot.

In certain embodiments of the present disclosure, the solid substrate is a microarray. An “array” or “microarray” is used synonymously herein to refer to a plurality of primers or probes attached to one or more distinguishable spots on a substrate. A microarray may include a single substrate or a plurality of substrates, for example a plurality of beads or microspheres. A “copy” of a microarray contains the same types and arrangements of primer or probes.

### Methods for Detecting Cardiovascular Disease

Better risk assessment for cardiovascular disease is the first step toward more effective prevention. Those identified as being at higher risk (e.g., PPV of 69% for CHD incidence within three years) can be followed up promptly for further testing such as with coronary calcium or angiography, and more aggressive interventions. Conversely, those at lower risk (e.g., NPV of 99% for CHD incident within three years) can be re-tested periodically and monitored to ensure continued prevention due to the dynamic nature of DNA methylation. Compared to the integrated genetic-epigenetic model, overall, conventional risk factors-based calculators were considerably less sensitive, less generalizable, and also depicted a gender gap in performance. In contrast, the integrated genetic-epigenetic model described herein has the ability to capture and better understand the complex nature of CVD via three angles, genetics (inherited risk that is static), DNA methylation (acquired risk that is dynamic) and the genetic confounding of methylation signatures (i.e., G+M+GxM).

The present disclosure provides a method for determining whether a subject has a likelihood of having a CVD incidence within, for example, three years, by determining methylation status of a CpG dinucleotide repeat or CpG dinucleotide repeat motif region, where the methylation status of the CpG dinucleotide is associated with the incidence of CVD. However, the same principals apply to other windows of incidence as well as to the assessment of both the prevalence and incidence of a number of different types of CVD including, without limitation, CHD, stroke, arrhythmia, cardiac arrest, congestive heart failure, atherosclerotic cardiovascular disease (ASCVD) and its associated cardiovascular events (CVE) including, for example, obstructive coronary artery disease (CAD), myocardial infarction (MI), stroke, and cardiovascular death (CVD). In certain embodiments, the method determines the methylation status of a plurality (e.g., any integer between 1 and 10,000, such as at least 100) of CpG dinucleotides and/or SNPs.

As used herein, a “biological sample” encompasses essentially any sample type obtained from a subject that can be used in a method as described herein. The biological sample may be any bodily fluid, tissue or any other sample from which clinically relevant biomarker levels may be determined. “Biological samples” also can encompasses cells in culture, cell supernatants, cell lysates, blood, serum, plasma, urine, cerebral spinal fluid, biological fluid, and tissue samples. Various techniques and reagents find use in the methods of the present disclosure. In one embodiment of the disclosure, blood samples, or samples derived from blood, e.g. plasma, circulating, peripheral, lymphocytes, etc., are assayed for the presence of one or more SNPs and/or the methylation status of one or more CpG dinucleotides. A biological sample also can be saliva. Typically, a biological sample that contains nucleic acids is provided and tested. Biological samples can be derived from subjects using well known techniques such as finger prick, venipuncture, lumbar puncture, fluid sample such as saliva or urine, or tissue biopsy and the like.

As used herein, the term “healthy” means that a subject does not manifest a particular condition, and is no more likely than at random to be susceptible to a particular condition.

Prevalence is defined by the American Psychological Association (APA) as the “the total number or percentage of cases (e.g., of a disease or disorder) existing in a population” (APA Dictionary of Psychology, (American Psychological Association, Washington, DC, 2007)). In some instances, point prevalence is used to describe the prevalence of cases at a discrete point of time, and period prevalence is used to describe the number of cases that exist for a period of time (e.g., a month, a year). Prevalence typically is expressed as a rate per population unit (e.g., number of cases per 100,000 people) instead of an absolute number or a percent.

Similarly, incidence is defined by the APA as “the rate of occurrence of new cases of a given event or condition (e.g., a disorder, disease, symptom, or injury) in a particular population in a given period” of time (APA Dictionary of Psychology, (American Psychological Association, Washington, DC, 2007)). As used herein, the term “incidence” is defined as a tendency or susceptibility for a subject to manifest a condition, in this case, CVD (e.g., CHD). In some instances, the period of time can be a year or less than a year; in some instances, the period of time can be longer than a year (e.g., two years, five years, ten years).

Diagnosis is defined by the APA as the “process of identifying and determining the nature of a disease or disorder by its signs and symptoms, through the use of assessment techniques (e.g., tests and examinations) and other available evidence” (APA Dictionary of Psychology, (American Psychological Association, Washington, DC, 2007)). A diagnosis can refer to the present time period, or to a time period in the past or the future.

Likewise, prognosis is defined by the APA as “a prediction of the course, duration, severity, and outcome of a condition, disease, or disorder” (APA Dictionary of Psychology, (American Psychological Association, Washington, DC, 2007)). A prognosis can be made, for example, over a period of one month, six months, one year, five years, ten years, or longer.

Risk assessment is defined as a “study of a subject done for the purpose of trying to determine the probability that that person will develop a particular disease or, if the disease is already present, the probability that the person will suffer exacerbation of it or death from it” (Youngson, 2005, Collins Dictionary of Medicine). In some instances, risk assessment is based on conditions or events and not on disease. In some instances, a risk assessment is determined over a period of time (e.g., months, years).

Biomarkers are described herein that can be used in methods (e.g., predictive or prognostic) of detecting the incidence (e.g., one-year, three-year, five-year) of CVD in a subject. Such methods typically include providing a biological sample from the subject; contacting DNA from the biological sample with bisulfite under alkaline conditions;

contacting the bisulfite-treated DNA with at least one first oligonucleotide primer at least 8 nucleotides in length that is complementary to a sequence that comprises a CpG dinucleotide (e.g., at a GC locus referred to as cg00300879, cg09552548, and cg14789911, or another biomarker from Appendix A); and determining the methylation status of the CpG dinucleotide. It would be understood that the at least one first oligonucleotide probe can detect either the unmethylated CpG dinucleotide or the methylated CpG dinucleotide. Such a method can further include determining the genotype of a single nucleotide polymorphism (SNP) (e.g., rs11716050, rs6560711, rs3735222, rs6820447, and rs9638144, or another biomarker from Appendix C) or a second SNP in linkage disequilibrium with the first SNP. As described herein, methylation of one or more particular CpG dinucleotides and the presence of one or more particular SNPs can be used to predict the three-year incidence of CHD in the subject.

In some embodiments, the method further comprises contacting the bisulfite-treated DNA with at least one second oligonucleotide probe at least 8 nucleotides in length that is complementary to a sequence that comprises a CpG dinucleotide, where the at least one second oligonucleotide probe detects either the unmethylated CpG dinucleotide or the methylated CpG dinucleotide, whichever is not detected by the at least one first oligonucleotide probe.

In some embodiments, the ratio of methylated CpG dinucleotides to unmethylated CpG dinucleotides in the biological sample can be determined as a part of the methods described herein. Determining the ratio of methylated CpG dinucleotides to unmethylated CpG dinucleotides can allow for a risk or outcome to be estimated or determined.

It would be appreciated that determining the methylation status of the one or more CpG dinucleotides and determining the presence (or absence) of a SNP can utilize any number of techniques, such as, for example, amplifying and/or sequencing steps. Amplifying and sequencing are well known techniques in the art and are used routinely to determine both the methylation status of a particular sequence and the presence/absence of a SNP.

Methods of determining the presence of biomarkers associated with the three-year incidence of CHD in a biological sample from a subject are provided. A similar approach can be used for any other form of CVD as well. Such methods typically include providing a first portion of the biological sample and contacting DNA from the first portion with bisulfite under alkaline conditions. The bisulfite-treated first portion can be contacted with a first oligonucleotide probe that is at least 8 nucleotides in length and that is complementary to a sequence that comprises a CpG dinucleotide (detected, e.g., at a CG locus referred to as cg00300879, cg09552548, and cg14789911, or another biomarker from Appendix A), and a second portion of the biological sample can be contacted with a nucleic acid probe at least 8 nucleotides in length that is complementary to a SNP (e.g., rs11716050, rs6560711, rs3735222, rs6820447, and rs9638144, or another biomarker from Appendix C).

As described herein, the percentage of methylation of the CpG dinucleotide at one or more of the GC loci designated cg00300879, cg09552548, and cg14789911 (or at a CpG dinucleotide that is in linkage disequilibrium with one or more of such CpG dinucleotides) and the identity of the nucleotide at one or more SNPs designated rs11716050, rs6560711, rs3735222, rs6820447, and rs9638144 (or at a SNP that is in linkage disequilibrium with one or more of such SNPs) are biomarkers associated with CVD and can be used to predict the likelihood that an individual will develop CVD and/or prognosticate as to the severity of the disease or the outcome for the individual.

While the effects of the indicated loci on whether or not an individual will develop CVD is a complex relationship, the following trends can be associated with CVD: decreasing methylation of the CpG dinucleotide at loci cg09552548 or cg14789911; increasing methylation of the CpG dinucleotide at loci cg00300879; the presence of a G nucleotide at SNP rs11716050, the presence of a G nucleotide at SNP rs6560711, the presence of a G nucleotide at SNP rs3735222, the presence of a C nucleotide at SNP rs6820447, and the presence of a G nucleotide at SNP rs9638144.

In addition to the SNP and CpG biomarkers identified herein, one or more clinical indicators can be used to aid in either or both diagnostics and prognostics. Without limitation, such clinical indicators can include demographics (e.g., age, sex, race); vital signs (e.g., heart rate (beats/min), systolic BP (mm Hg), diastolic BP (mm Hg)); medical history (e.g., smoking, atrial fibrillation/flutter, hypertension, coronary heart disease, myocardical infarction, heart failure, peripheral artery disease, COPD, diabetes (type 1 or type 2), CVA/TIA, chronic kidney disease, hemodialysis, angioplasty (peripheral or coronary), stent (peripheral or coronary), CABG, percutaneous coronary intervention); medications (ACE-I/ARB, beta blocker, aldosterone antagonist, loop diuretics, nitrates, CCB, statin, aspririn, warfarin, clopidogrel); echocardiographic results (e.g., LVEF (%), RSVP (mm Hg)); stress test results (e.g., ischemia on scan, ischemia on ECG); angiography results (e.g., ≥70% coronary stenosis in ≥2 vessels, ≥70% coronary stenosis in ≥3 vessels); and/or lab measures (e.g., sodium, blood urea nitrogen (mg/dL), creatinine (mg/dL), eGFR (median, CKDEPI), total cholesterol (mg/dL), LDL cholesterol (mg/dL), glycohemoglobin (%), glucose (mg/dL), HGB (mg/dL)).

### Kits for Detecting Cardiovascular Disease

In a further embodiment of the disclosure, articles of manufacture and kits containing probes, oligonucleotides or antibodies are provided. Such articles of manufacture can be used in the methods described herein. An article of manufacture can include one or more containers with, for example, a label. Suitable containers include, for example, bottles, vials, and test tubes. The containers can be formed from a variety of materials such as glass or plastic. The container can hold a composition that includes one or more agents that are effective for practicing the methods described herein. The label on the container indicates that the composition can be used for a specific application. The kit of the disclosure will typically comprise the container described above and one or more other containers comprising materials desirable from a commercial and user standpoint, including buffers, diluents, filters and package inserts with instructions for use.

In certain embodiments, the present disclosure provides a kit for determining the methylation status of at least one CpG dinucleotide and the presence of at least one single-nucleotide polymorphism (SNP). In certain embodiments, a kit as described herein may contain a number of primers that is any integer between 1 and 10,000, such as 1, 2, 3, 4, 5, 6, 7, 8, 9, 10, . . . 9997, 9998, 9999, 10,000. As used herein, the term “nucleic acid primer” or “nucleic acid probes” or “oligonucleotide” encompasses both DNA and RNA sequences. In certain embodiments, the primers or probes may be physically located on a single solid substrate or on multiple substrates.

A kit as described herein can include at least one first nucleic acid primer (e.g., at least 8 nucleotides in length) that is complementary to a bisulfite-converted nucleic acid sequence comprising a CpG dinucleotide (detected, e.g., at a GC locus referred to as cg00300879, cg09552548, and cg14789911), and at least one second nucleic acid primer (e.g., at least 8 nucleotides in length) that is complementary to a SNP (e.g., rs11716050, rs6560711, rs3735222, rs6820447, and rs9638144). The at least one first nucleic acid primer can detect the methylated or unmethylated CpG dinucleotide.

It would be appreciated that any of the nucleic acid primers, probes or oligonucleotides described herein can include one or more nucleotide analogs and/or one or more synthetic or non-natural nucleotides.

It also would be appreciated that the kits described herein can include a solid substrate. In some embodiments, one or more of the nucleic acid primers can be bound to the solid support. Examples of solid supports include, without limitation, polymers, glass, semiconductors, papers, metals, gels or hydrogels. Additional examples of solid supports include, without limitation, microarrays or microfluidics cards.

It also would be appreciated that any of the kits described herein can include one or more detectable labels. In some embodiments, one or more of the nucleic acid primers can be labeled with the one or more detectable labels. Representative detectable labels include, without limitation, an enzyme label, a fluorescent label, and a colorimetric label.

### Algorithm for Predicting the Incidence of Cardiovascular Disease

Any number of algorithms that can capture linear effects (e.g., linear regression) or both linear and non-linear effects (e.g., Random Forest, Gradient Boosting, Neural Networks (e.g., deep neural network, extreme learning machine (ELM)), Support Vector Machine, Hidden Markov model) can be used in the methods described herein. See, for example, Mckinney et al., 2011, Appl. Bioinform., 5(2):77-88; Gunther et al., 2012, BMC Genet., 13:37; and Ogutu et al., 2011, BMC Proceedings, 5(Suppl 3): S11. Any type of machine learning algorithm or deep learning neural network algorithm (tuned or non-tuned) capable of capturing linear and/or non-linear contribution of traits for the prediction can be used. In some instances, a combination of algorithms (e.g., a combination or ensemble of multiple algorithms that capture linear and/or non-linear contributions of traits) is used.

Simply by way of example, Random Forest™ is a popular machine learning algorithm created by Breiman & Cutler for generating “classification trees” (see, for example, “stat.berkeley.edu/˜breiman/RandomForests/cc_home.htm” on the World Wide Web). Using standard machine learning and predictive modeling techniques, a diagnostic classifier algorithm was written to be implemented in R and Python programming languages (though it can be implemented in many other programming languages), according to well described guidelines by Breiman & Cutler. A diagnostic classifier algorithm was generated using data from at least two traits (T) and the diagnosis of interest from that population. To determine the output (e.g., diagnosis) for a new individual, one simply determines values for the at least two traits (T) and inputs that information into an algorithm (e.g., the diagnostic classifier algorithm described herein or another algorithm discussed above) that is capable of capturing the linear and non-linear contributions of the traits.

As described herein, the inputs are at least one genotype (e.g., SNP) and the methylation status of at least one CpG dinucleotide, and the outcome can represent a positive or a negative probability for the incidence (e.g., one-year, three-year, five-year) of CVD. The Traits (T) used to determine the outcome can represent the methylation status of at least one CpG dinucleotide or at least one genotype (e.g., of a SNP), but Traits (T) also can correspond to at least one interaction (e.g., between methylation status and genotype (CpGxSNP), between the methylation status of two different sites (CpGxCpG) or between two different genotypes (SNPxSNP)). It would be appreciated that any such interactions can be visualized using partial dependence plots.

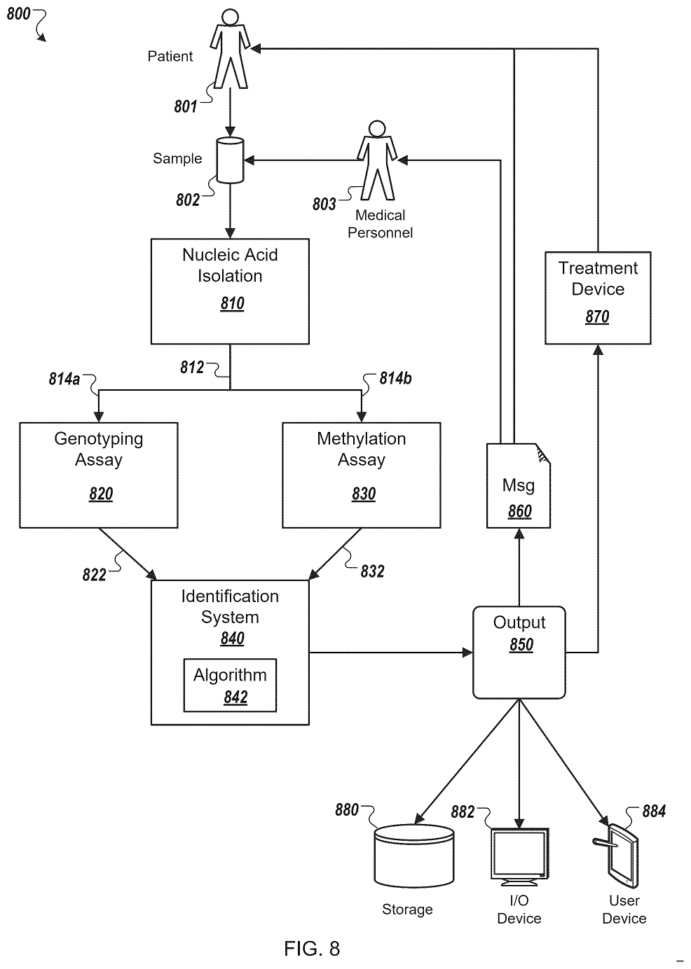

FIG. 8 is a block diagram of an example coronary heart disease classification system 800. In some embodiments, the system 800 can perform monitoring and/or prediction of coronary heart disease. For example, the system 800 can be used to perform one or more of the example processes described herein.

In the illustrated example, a subject 801 provides a subject sample 802. In some embodiments, the subject sample 802 can be a blood sample, a saliva sample, a mucus sample, a urine or stool sample, or any other appropriate biological sample from the subject 801. In some embodiments, medical personnel 803 (e.g., a doctor, a nurse, a lab technician, a caregiver) may assist the subject 801 with obtaining the subject sample 802. In some embodiments, the subject 801 may obtain the subject sample 802 from herself or himself (e.g., by using a portable blood sampling device or a home collection kit).

A nucleic acid isolation module 810 isolates a nucleic acid sample 812 from the subject sample 802. In some embodiments, the nucleic acid isolation module 810 can be a manual, semi-automated, or automatic process that perform or more of cell lysis, removal of contaminating proteins, deactivating DNAases and/or RNAases, and recovery of DNA and/or RNA. For example, the nucleic acid isolation module 810 can be a part of an automated process or analysis device configured to isolate the nucleic acid sample 812 from the subject sample 802. In another example, the nucleic acid isolation module 810 can be part of one or more of the example kits described in this document, to be used by a human user such as the medical personnel 803.

A genotyping assay module 820 receives a portion 814a of the nucleic acid sample 812. The genotyping assay module 820 is configured to perform a genotyping assay on the portion 814a of the nucleic acid sample 812 to detect the presence of at least one SNP, wherein the at least one SNP is a first SNP from Appendix C and/or is a second SNP in linkage disequilibrium (R>0.3) with a first SNP from Appendix C to determine, identify, or otherwise obtain a collection of genotype data 822. In some embodiments, the genotyping assay module 820 can be a manual, semi-automated, or automatic process. For example, genotyping assay module 820 can be a part of an automated process or analysis device configured to perform a genotyping assay on the portion 814a. In another example, the genotyping assay module 820 can be part of one or more of the example kits described in this document, to be used by a human user such as the medical personnel 803 or a laboratory technician.

A methylation assay module 830 receives a portion 814b of the nucleic acid sample 812. The methylation assay module 830 is configured to bisulfite convert the nucleic acid in the portion 814b of the nucleic acid sample 812 and perform methylation assessment on the portion 814b of the nucleic acid sample 812 to detect methylation status of at least one CpG site from Appendix A and/or a CpG site collinear (R>0.3) with a CpG from Appendix A to determine, identify, or otherwise obtain a collection of methylation data 832.

An identification system 840 is configured to receive the collection of genotype data 822 and the collection of methylation data 832, and identify one or more predetermined traits or characteristics of the subject 801 based on a diagnostic classifier algorithm module 842. The diagnostic classifier algorithm module 842 is configured to account for at least one SNP main effect and/or at least one CpG main effect and/or at least one interaction effect. In some embodiments, the diagnostic classifier algorithm module 842 can perform one or more of the algorithms described herein that may indicate the presence of disease (e.g., diagnostic indicators) or a propensity to develop disease (e.g., predict). For example, the identification system may be configured to identify genetic and/or environmental characteristics that determines the presence of or the likelihood of a subject developing disease (e.g., cardiovascular disease), even when the disease is of polygenic origin. In some implementations, the diagnostic classifier algorithm module 842 can be a machine learning algorithm capable of accounting for linear and non-linear effects.

The identification system 840 provides an output 850 based on the diagnostic and/or prognostic indicators provided by the diagnostic classifier algorithm module 842. In some embodiments, the identification system 840 can include an output module configured to provide the output 850. In some implementations, the output 850 can be an identification of one or more diseases that the subject 801 may already have. For example, the output 850 may indicate that traits that are indicative of the presence of cardiovascular disease were found in the subject 801. In some implementations, the output 850 can be an indication of a likelihood that the subject 801 may develop a disease within a predetermined time frame (e.g., the subject 801 may have a 43% chance of developing cardiovascular disease within 3 years, the subject 801 may have a 77% of having a heart attack within 2 years). In some implementations, the output 850 can include therapeutic and/or preventative recommendations based on the diagnostic and/or prognostic indicators provided by the diagnostic classifier algorithm module 842. For example, in response to an identification or prediction of a diabetic or cardiac condition in the subject 801, the output 850 may include a recommendation to consult with the medical personnel 803, identify possible dietary or lifestyle changes by the subject 801 to address or avoid the condition, identify potential treatments and/or remedies for the subject 801 to consider in consultation with the medical personnel 803, or combinations of these and/or any other appropriate information based on the output of the algorithm(s) of the diagnostic classifier algorithm module 842.

In the illustrated example, the output 850 is provided in various formats. The information provided by the output 850 can be formatted into a message 860 that is provided to the subject 801 and/or to the medical personnel 803. In some implementations, the message 860 can be formatted as a report (e.g., a word processing file, a portable document format file) that is at least temporarily stored on a non-transitory storage medium (e.g., a hard drive, a FLASH memory), where it can be retrieved by the subject 801 and/or the medical personnel 803 for review. In some implementations, the message 860 can be formatted as an electronic message (e.g., an email, a text message, an instant message) that is transmitted to the subject 801 and/or the medical personnel 803 for review. In some implementations, the message 860 can be a printed report. For example, the output 850 can be provided to a printing system that is configured to generate a hard copy report based on the output 850. Subsequent automated or manual processing systems can package the report as a letter or other parcel that can be sent for physical delivery to the subject 801 and/or to the medical personnel 803 (e.g., the system 800 can created a paper printout the results and mail them through postal mail).

A treatment device 870 can be configured to receive the diagnostic and/or prognostic indicators provided by the output 850 and provide therapy and/or treatment based on the diagnostic and/or prognostic indicators. For example, the output 850 may indicate that the subject 801 has a high likelihood of suffering cardiac arrest within the next two years, and the treatment device 870 may be a drug (e.g., a tablet or capsule) or an implantable drug delivery system that reacts by identifying or by receiving configuration settings for an appropriate dosage of a statin, acetylsalicylic acid (aspirin), an anti-inflammatory drug, a blood thinner, or combinations of these and/or any other appropriate therapeutic and/or preventative substances. In some embodiments, the treatment device 870 can be configured to also include one or more of the nucleic acid isolation module 810, the genotyping assay module 820, the methylation assay module 830, or the identification system 840.

A storage system 880 is configured to store the output 850. For example, the information included in the output 850 can be stored temporarily, for a predetermined period of time, or substantially permanently in a database, in a file, or as any other appropriate collection of data. In some embodiments, the storage system 880 can store the output 850 in a non-transitory storage medium (e.g., a hard drive, a FLASH memory). For example, the output 850 may include some or all of the collection of genotype data 822, the collection of methylation data 832, and/or the output 850 in personal health record that the subject 801 can store or carry with them. In some embodiments, the storage system 880 can store the output 850 as a physical medium, for example, the storage system 880 can include a printer that can generate a paper report based on the output 850, and/or store the report as a hard copy that can be physically filed away for later retrieval.

An input/output device 882 is physical device configured to display or otherwise present an output that is perceptible to humans (e.g., the subject 801, the medical personnel 803). For example, the input/output device 882 may be an electronic display device in a doctor's office. The system 800 may process the subject sample 802, and then alter the configuration of pixels onscreen to modify the information displayed by the input/output device 882 based on the output 850 (e.g., a screen can be updated to display an identified diagnosis and/or prognosis for the subject 801 to the medical personnel). In another example, the input/output device 882 can be configured to provide audible (e.g., spoken output) and/or tactile (e.g., braille, haptic, vibratory) output that modifies or otherwise transforms the output 850 into a physical and/or tangible output (e.g., to convey the diagnostic and/or prognostic indicators in a manner that is perceptible to a user who is sight-challenged). In another example, the input/output device 882 can be configured to alter, transform, or modify a physical characteristic of a physical structure or medium based on the output 850.

A user device 884 (e.g., a computer, a smartphone, a tablet computer, a computerized terminal) is configured to display, emit, or otherwise present one or more outputs that are perceptible to a human user, such as the subject 801 and/or the medical personnel 803. For example, the user device 884 can receive the output 850 (e.g., as data, as the message 860) and provide an alert to the user and/or provide an output (e.g., display a report, read a report aloud) based on the output 850. In some embodiments, the user device 884 can include one or more of the storage device 880 or the input/output device 882. In some embodiments, the user device 882 can be part of the treatment device 870. In some embodiments, the user device 884 can be configured to include one or more of the nucleic acid isolation module 810, the genotyping assay module 820, the methylation assay module 830, or the identification system 840.

In some implementations, some or all of the system 800 may be reused to provide additional information. For example, the system 800 may be used to gather an initial set of health information for the subject 801 and/or identify information that can assist the medical personnel 803 with an initial diagnosis/prognosis. Later, the patent 801 may be re-examined using the system 800, for example, to determine the effectiveness of prescribed medical and/or lifestyle strategies over time. Since the collection of genotype data 822 does not change over time for an individual person, the system 800 may refrain from performing the functions of the genotyping assay module 820 again. In such examples, the methylation assay module 830 may be used to generate an updated version of the collection of methylation data 832, and the updated collection of methylation data 832 can be provided to the identification system 840 for processing along with the collection of genotype data 822 that was previously generated. In some implementations, the subject sample 802 can be collected on a periodic basis and processed based on the existing collection of genotype data 822 and updated collections of methylation data 832 to produce updated outputs 850 that can be used to provide ongoing monitoring of one or more conditions identified for the subject 801.

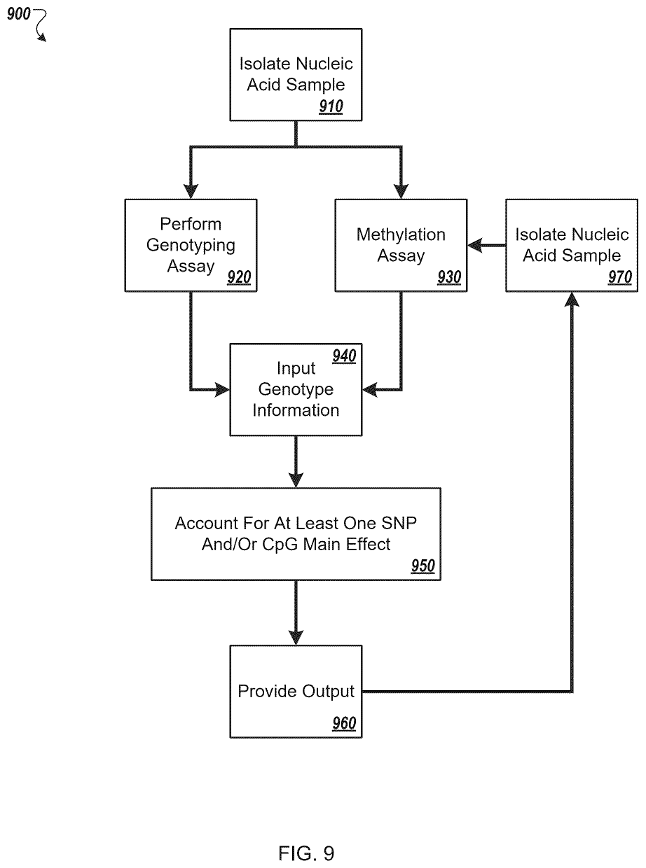

FIG. 9 is a flow diagram of an example process 900 for cardiovascular disease classification. In some implementations, the process 900 can be some or all of the example processes described above. In some implementations, the process 900 can be the process performed by some or all of the example system 800 of FIG. 8.

At 910, a nucleic acid sample is isolated from a subject sample. For example, the example nucleic acid isolation module 810 can be configured to isolate and/or substantially purify nucleic acid compositions from the example subject sample 802 to produce the example nucleic acid sample 812.

At 920, a genotyping assay is performed on a first portion of the nucleic acid sample to detect the presence of at least one SNP, wherein the at least one SNP is a first SNP from Appendix C and/or is a second SNP in linkage disequilibrium (R>0.3) with a first SNP from Appendix C to obtain genotype data. For example, the example genotyping assay module 820 could be used to analyze the example portion 814a of the nucleic acid sample 812 to produce the example collection of genotype data 822.

At 930, a second portion of the nucleic acid sample is bisulfite converted, and a methylation assessment is performed on the second portion of the nucleic acid sample to detect methylation status of at least one CpG site from Appendix A and/or a CpG site collinear (R>0.3) with a CpG from Appendix A to obtain methylation data. For example, the example methylation assay module 830 can be used to process the portion 814b of the nucleic acid sample 812 to produce the example collection of methylation data 832.

At 940, the genotype data from step 920 and/or methylation data from step 930 is input into an algorithm. For example, the example collection of genotype data 822 and the example collection of methylation data 832 are input into the example identification system 840 and processed using the example diagnostic classifier algorithm module 842.

At 950, at least one SNP main effect and/or at least one CpG main effect and/or at least one interaction effect are accounted for. For example, the example diagnostic classifier algorithm module 842 can be configured to account for at least one SNP main effect and/or at least one CpG main effect and/or at least one interaction effect. In some implementations, the diagnostic classifier algorithm module 842, can be a machine learning algorithm capable of accounting for linear and non-linear effects.

At 960, an output is provided. For example, the example identification system 840 can provide the example output 850.

At 970 another nucleic acid sample is isolated from another sample from the subject. For example, the example nucleic acid isolation module 810 can be configured to isolate and/or substantially purify nucleic acid compositions from another sample to produce another example nucleic acid sample. Since the collection of genotype data 822 from a subject does not change over time, the newly-produced nucleic acid sample can be used to obtain methylation data 832, which is used along with the existing collection of genotype data 822 to provide an updated output (e.g., to perform a checkup on the subject 801 at a later point in time). In some implementations, this abbreviated process can be performed on a periodic or semi-periodic basis to provide ongoing monitoring of one or more medical conditions identified for the subject 801.

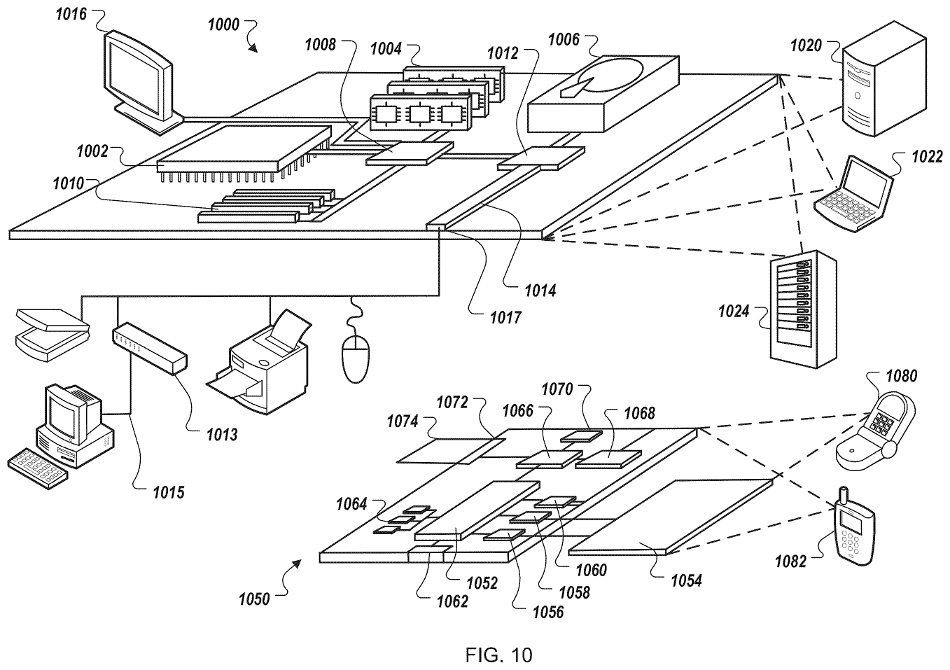

FIG. 10 is a block diagram of example computing devices 1000, 1050 that may be used to implement the systems and methods described in this document, either as a client or as a server or plurality of servers. Computing device 1000 is intended to represent various forms of digital computers, such as laptops, desktops, workstations, personal digital assistants, servers, blade servers, mainframes, and other appropriate computers. Computing device 1000 can also represent all or parts of various forms of computerized devices, such as embedded digital controllers, media bridges, modems, network routers, network access points, network repeaters, and network interface devices including mesh network communication interfaces. Computing device 1050 is intended to represent various forms of mobile devices, such as personal digital assistants, cellular telephones, smartphones, and other similar computing devices. The components shown here, their connections and relationships, and their functions, are meant to be exemplary only, and are not meant to limit implementations of the compositions and methods described herein.

Computing device 1000 includes a processor 1002, a memory 1004, a storage device 1006, a high-speed interface 1008 connecting to memory 1004 and high-speed expansion ports 1010, and a low speed interface 1012 connecting to a low speed bus 1014 and storage device 1006. Each of the components 1002, 1004, 1006, 1008, 1010, and 1012, are interconnected using various busses, and may be mounted on a common motherboard or in other manners as appropriate. The processor 1002 can process instructions for execution within the computing device 1000, including instructions stored in the memory 1004 or on the storage device 1006 to display graphical information for a GUI on an external input/output device, such as display 1016 coupled to high speed interface 1008. In other implementations, multiple processors and/or multiple buses may be used, as appropriate, along with multiple memories and types of memory. Also, multiple computing devices 1000 may be connected, with each device providing portions of the necessary operations (e.g., as a server bank, a group of blade servers, or a multi-processor system).

The memory 1004 stores information within the computing device 1000. In one implementation, the memory 1004 is a computer-readable medium. In one implementation, the memory 1004 is a volatile memory unit or units. In another implementation, the memory 1004 is a non-volatile memory unit or units.

The storage device 1006 is capable of providing mass storage for the computing device 1000. In one implementation, the storage device 1006 is a computer-readable medium. In various different implementations, the storage device 1006 may be a floppy disk device, a hard disk device, an optical disk device, or a tape device, a flash memory or other similar solid state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer-or machine-readable medium, such as the memory 1004, the storage device 1006, or memory on processor 1002.

The high speed controller 1008 manages bandwidth-intensive operations for the computing device 1000, while the low speed controller 1012 manages lower bandwidth-intensive operations. Such allocation of duties is exemplary only. In one implementation, the high-speed controller 1008 is coupled to memory 1004, display 1016 (e.g., through a graphics processor or accelerator), and to high-speed expansion ports 1010, which may accept various expansion cards (not shown). In the implementation, low-speed controller 1012 is coupled to storage device 1006 and low-speed expansion port 1017 through the low-speed bus 1014. The low-speed expansion port, which may include various communication ports (e.g., Universal Serial Bus (USB), BLUETOOTH, BLUETOOTH Low Energy (BLE), Ethernet, wireless Ethernet (WiFi), High-Definition Multimedia Interface (HDMI), ZIGBEE, visible or infrared transceivers, Infrared Data Association (IrDA), fiber optic, laser, sonic, ultrasonic) may be coupled to one or more input/output devices, such as a keyboard, a pointing device, a scanner, a networking device such as a gateway, modem, switch, or router, e.g., through a network adapter 1013.

Peripheral devices can communicate with the high speed controller 1008 through one or more peripheral interfaces of the low speed controller 1012, including but not limited to a USB stack, an Ethernet stack, a WiFi radio, a BLUETOOTH Low Energy (BLE) radio, a ZIGBEE radio, an HDMI stack, and a BLUETOOTH radio, as is appropriate for the configuration of the particular sensor. For example, a sensor that outputs a reading over a USB cable can communicate through a USB stack.

The network adapter 1013 can communicate with a network 1015. Computer networks typically have one or more gateways, modems, routers, media interfaces, media bridges, repeaters, switches, hubs, Domain Name Servers (DNS), and Dynamic Host Configuration Protocol (DHCP) servers that allow communication between devices on the network and devices on other networks (e.g. the Internet). One such gateway can be a network gateway that routes network communication traffic among devices within the network and devices outside of the network. One common type of network communication traffic that is routed through a network gateway is a Domain Name Server (DNS) request, which is a request to the DNS to resolve a uniform resource locator (URL) or uniform resource indicated (URI) to an associated Internet Protocol (IP) address.

The network 1015 can include one or more networks. The network(s) may provide for communications under various modes or protocols, such as Global System for Mobile communication (GSM) voice calls, Short Message Service (SMS), Enhanced Messaging Service (EMS), or Multimedia Messaging Service (MMS) messaging, Code Division Multiple Access (CDMA), Time Division Multiple Access (TDMA), Personal Digital Cellular (PDC), Wideband Code Division Multiple Access (WCDMA), CDMA2000, General Packet Radio System (GPRS), or one or more television or cable networks, among others. For example, the communication may occur through a radio-frequency transceiver. In addition, short-range communication may occur, such as using a BLUETOOTH, BLE, ZIGBEE, WiFi, IrDA, or other such transceiver.

In some embodiments, the network 1015 can have a hub-and-spoke network configuration. A hub-and-spoke network configuration can allow for an extensible network that can accommodate components being added, removed, failing, and replaced. This can allow, for example, more, fewer, or different devices on the network 1015. For example, if a device fails or is deprecated by a newer version of the device, the network 1015 can be configured such that network adapter 1013 can to be updated about the replacement device.

In some embodiments, the network 1015 can have a mesh network configuration (e.g., ZIGBEE). Mesh configurations may be contrasted with conventional star/tree network configurations in which the networked devices are directly linked to only a small subset of other network devices (e.g., bridges/switches), and the links between these devices are hierarchical. A mesh network configuration can allow infrastructure nodes (e.g., bridges, switches and other infrastructure devices) to connect directly and non-hierarchically to other nodes. The connections can be dynamically self-organize and self-configure to route data. By not relying on a central coordinator, multiple nodes can participate in the relay of information. In the event of a failure of one or more of the nodes or the communication links between then, the mesh network can self-configure to dynamically redistribute workloads and provide fault-tolerance and network robustness.

The computing device 1000 may be implemented in a number of different forms, as shown in the figure. For example, it may be implemented as a standard server 1020, or multiple times in a group of such servers. It may also be implemented as part of a rack server system 1024. It may also be implemented as part of network device such a modem, gateway, router, access point, repeater, mesh node, switch, hub, or security device (e.g., camera server). In addition, it may be implemented in a personal computer such as a laptop computer 1022. Alternatively, components from computing device 1000 may be combined with other components in a mobile device (not shown), such as device 1050. In some embodiments, the device 1050 can be a mobile telephone (e.g., a smartphone), a handheld computer, a tablet computer, a network appliance, a camera, an enhanced general packet radio service (EGPRS) mobile phone, a media player, a navigation device, an email device, a game console, an interactive or so-called “smart” television, a media streaming device, or a combination of any two or more of these data processing devices or other data processing devices. In some implementations, the device 1050 can be included as part of a motor vehicle (e.g., an automobile, an emergency vehicle (e.g., fire truck, ambulance), a bus). Each of such devices may contain one or more of computing device 1000, 1050, and an entire system may be made up of multiple computing devices 1000, 1050 communicating with each other through a low speed bus or a wired or wireless network.

Computing device 1050 includes a processor 1052, memory 1064, an input/output device such as a display 1054, a communication interface 1066, and a transceiver 1068, among other components. The device 1050 may also be provided with a storage device, such as a microdrive or other device, to provide additional storage. Each of the components 1050, 1052, 1064, 1054, 1066, and 1068, are interconnected using various buses, and several of the components may be mounted on a common motherboard or in other manners as appropriate.

The processor 1052 can process instructions for execution within the computing device 1050, including instructions stored in the memory 1064. The processor may also include separate analog and digital processors. The processor may provide, for example, for coordination of the other components of the device 1050, such as control of user interfaces, applications run by device 1050, and wireless communication by device 1050.

Processor 1052 may communicate with a user through control interface 1058 and display interface 1056 coupled to a display 1054. The display 1054 may be, for example, a TFT LCD display or an OLED display, or other appropriate display technology. The display interface 1056 may comprise appropriate circuitry for driving the display 1054 to present graphical and other information to a user. The control interface 1058 may receive commands from a user and convert them for submission to the processor 1052. In addition, an external interface 1062 may be provide in communication with processor 1052, so as to enable near area communication of device 1050 with other devices. External interface 1062 may provide, for example, for wired communication (e.g., via a docking procedure) or for wireless communication (e.g., via Bluetooth or other such technologies).

The memory 1064 stores information within the computing device 1050. In one implementation, the memory 1064 is a computer-readable medium. In one implementation, the memory 1064 is a volatile memory unit or units. In another implementation, the memory 1064 is a non-volatile memory unit or units. Expansion memory 1074 may also be provided and connected to device 1050 through expansion interface 1072, which may include, for example, a SIMM card interface. Such expansion memory 1074 may provide extra storage space for device 1050, or may also store applications or other information for device 1050.

Specifically, expansion memory 1074 may include instructions to carry out or supplement the processes described above, and may include secure information also. Thus, for example, expansion memory 1074 may be provide as a security module for device 1050, and may be programmed with instructions that permit secure use of device 1050. In addition, secure applications may be provided via the SIMM cards, along with additional information, such as placing identifying information on the SIMM card in a non-hackable manner.

The memory may include for example, flash memory and/or MRAM memory, as discussed below. In one implementation, a computer program product is tangibly embodied in an information carrier. The computer program product contains instructions that, when executed, perform one or more methods, such as those described above. The information carrier is a computer-or machine-readable medium, such as the memory 1064, expansion memory 1074, or memory on processor 1052.

Device 1050 may communicate wirelessly through communication interface 1066, which may include digital signal processing circuitry where necessary. Communication interface 1066 may provide for communications under various modes or protocols, such as GSM voice calls, Voice Over LTE (VOLTE) calls, SMS, EMS, or MMS messaging, CDMA, TDMA, PDC, WCDMA, CDMA2000, GPRS, WiMAX, LTE, 5G, among others. Such communication may occur, for example, through radio-frequency transceiver 1068. In addition, short-range communication may occur, such as using a Bluetooth, WiFi, or other such transceiver (not shown) configured to provide uplink and/or downlink portions of data communication. In addition, GPS receiver module 1070 may provide additional wireless data to device 1050, which may be used as appropriate by applications running on device 1050.

Device 1050 may also communication audibly using audio codec 1060, which may receive spoken information from a user and convert it to usable digital information. Audio codex 1060 may likewise generate audible sound for a user, such as through a speaker, e.g., in a handset of device 1050. Such sound may include sound from voice telephone calls, may include recorded sound (e.g., voice messages, music files, etc.) and may also include sound generated by applications operating on device 1050.

The computing device 1050 may be implemented in a number of different forms, as shown in the figure. For example, it may be implemented as a cellular telephone 1080. It may also be implemented as part of a smartphone 1082, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

Some communication networks can be configured to carry power as well as information on the same physical media. This allows a single cable to provide both data connection and electric power to devices. Examples of such shared media include power over network configurations in which power is provided over media that is primarily or previously used for communications. One specific embodiment of power over network is Power Over Ethernet (POE) which pass electric power along with data on twisted pair Ethernet cabling. Examples of such shared media also include network over power configurations in which communication is performed over media that is primarily or previously used for providing power. One specific embodiment of network over power is Power Line Communication (PLC) (also known as power-line carrier, power-line digital subscriber line (PDSL), mains communication, power-line telecommunications, or power-line networking (PLN), Ethernet-Over-Power (EOP)) in which data is carried on a conductor that is also used simultaneously for AC electric power transmission.

The computing system can include clients and servers. A client and server are generally remote from each other and typically interact through a communication network. The relationship of client and server arises by virtue of computer programs running on the respective computers and having a client-server relationship to each other.

The computing system can include routers, gateways, modems, switches, hub, bridges, and repeaters. A router is a networking device that forwards data packets between computer networks and performs traffic directing functions. A network switch is a networking device that connects networked devices together by performing packet switching to receive, process, and forward data to destination devices. A gateway is a network device that allows data to flow from one discrete network to another. Some gateways can be distinct from routers or switches in that they can communicate using more than one protocol and can operate at one or more of the seven layers of the open systems interconnection model (OSI). A media bridge is a network device that converts data between transmission media so that it can be transmitted from computer to computer. A modem is a type of media bridge, typically used to connect a local area network to a wide area network such as a telecommunications network. A network repeater is a network device that receives a signal and retransmits it to extend transmissions and allow the signal can cover longer distances or overcome a communications obstruction.

It will be apparent that the present disclosure provides a skilled artisan the ability to construct a matrix in which the methylation status of one or more CpG dinucleotides and one or more genotypes (e.g., SNPs; e.g., at one or more alleles) can be evaluated as described herein, typically using a computer, to identify interactions and allow for prediction of the incidence of CVD. Although such an analysis is complex, no undue experimentation is required as all necessary information is either readily available to the skilled artisan or can be acquired by experimentation as described herein.

### Methods of Treating Cardiovascular Diseases

The present disclosure provides a method for determining the likelihood that a subject will have a CVD event within, for example, one year, three years, or five years. As used herein, CVD includes, without limitation, CHD, stroke, arrhythmia, cardiac arrest, and congestive heart failure. The methods and compositions described herein provide a better ability to assess a subjects risk for cardiovascular disease, which is the first step toward more effective prevention.

Upon making a positive prognosis of a cardiac outcome (e.g., a prognosis of cardiovascular death, myocardial infarct (MI), stroke, all cause death, or a composite thereof), a medical practitioner can advantageously use the prognostic information thereby obtained to identify the need for an intervention in the subject, such as, for example, stress testing with ECG response or myocardial perfusion imaging, coronary computed tomography angiogram, diagnostic cardiac catheterization, percutaneous coronary (e.g., balloon angioplasty with or without stent placement), coronary artery bypass graft (CABG), enrollment in a clinical trial, and administration or monitoring of effects of agents selected from, but not limited to, of agents selected from nitrates, beta blockers, ACE inhibitors, antiplatelet agents and lipid-lowering agents.

Those identified as being at higher risk (e.g., PPV of 69% for CHD incidence within three years) can be followed up promptly for further testing or more aggressive interventions. Conversely, those at lower risk can be re-tested periodically and monitored to ensure continued prevention due to the dynamic nature of DNA methylation.

Treatments for cardiovascular disease can depend on the type of cardiovascular disease and the symptoms the individual is experiencing. Treatments for cardiovascular disease can be preventative, therapeutic or palliative. Treatments for cardiovascular diseases can include, for example, lifestyle changes (e.g., diet (e.g., low fat diet), weight loss, exercise, reduction or cessation in smoking and/or drinking), pharmaceuticals (e.g., beta blockers, statins, calcium channel blocker, ACE inhibitors, vasodilator, alteplase) and/or surgical interventions (e.g., angioplasty, bypasss surgery, implantable device, endarterectomy).

In accordance with the present disclosure, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Materials and Methods

This study features data and/or biomaterial from two sources. The first set of anonymized genome-wide genetic, genome-wide DNA methylation and clinical data are from the eighth examination cycle of the Framingham Heart Study (FHS) Offspring cohort, while the second set of anonymized clinical data and DNA are from the Intermountain Healthcare (IM) biorepository. The procedures and protocols used for the analysis of the FHS data were approved by the University of Iowa Institutional Review Board (IRB # 201503802), and the procedures and protocols used for the analyses of the IM materials were approved by the Intermountain Healthcare Institutional Review Board (IRB #1024811).

### Example 2—Framingham Heart Study Offspring Cohort

The details on the collection and preparation of clinical and biological data of the FHS cohort have been described previously (dbGAP study accession: phs000007). In brief, the demographics, risk factors and clinical information were derived from the eighth examination of the Offspring cohort, with additional clinical follow-up information used to determine incident coronary heart disease (CHD) status. Incident CHD was considered present if an individual was diagnosed with CHD within three years of the eighth examination cycle. Conversely, incident CHD was considered absent if an individual was not diagnosed with CHD within three years of the eighth examination cycle. Data from those with prevalent CHD at the eighth examination cycle were excluded from further consideration. Sources of clinical data in determining incident CHD events included subject report, review of medical records, and death certificates. The designations and dates of CHD onset used in this study are as determined by a panel of three investigators on the Framingham Endpoint Review Committee.

Genome-wide DNA methylation data profiled using the Illumina Infinium HumanMethylation450 BeadChip array (San Diego, CA, USA) was available from 2,567subjects who were phlebotomized at the eighth examination cycle. Standard sample and probe level quality control were performed as described in previous studies, which resulted in retaining 2,560 samples and DNA methylation data from 403,192 loci (see, e.g., Dogan et al., 2018, Genes, 9:641; Pidsley et al., 2013, BMC Genomics, 14:1-10; Triche, 2014, FDb.InfiniumMethylation.hg19: Annotation package for Illumina Infinium DNA methylation probes. Vol. R package version 2.2.0; Davis et al., 2018, Handle Illumina methylation data., Vol. R package version 2.22.0; and Dogan et al., 2018, PLOS One, 13:e0190549). Genome-wide genotype data obtained using the Affymetrix GeneChip HumanMapping 500K array (Santa Clara, CA, USA) was available for 2,406 of the remaining samples. After standard sample and probe level quality control procedures were performed in PLINK on the array data as described previously, the total number of samples and SNPs remaining were 2,295 and 472,822, respectively (Dogan et al., 2018, Genes, 9:641; Dogan et al., 2018, PLOS One, 13:e0190549; and Purcell et al., 2007, Am. J. Hum. Genet., 81:559-75). A challenge in conducting biological studies of community cohorts such as the FHS is the potential for inter-relatedness of some of the subjects. Therefore, the genetic data were subjected to relatedness analysis in PLINK. Based on relatedness and incident CHD status, 1,280 subjects (18/542 males and 10/738 females diagnosed with clinical CHD within three years of the eighth examination cycle ascertainment) were part of the training set and 639 subjects (9/271 males and 5/368 females diagnosed with clinical CHD within three years of the eighth examination cycle ascertainment) were part of the test set. The demographics and conventional risk factors of these individuals are summarized in Table 1.

TABLE 1
Summary of demographics and conventional CHD risk factors for
the individuals in the Framingham Heart Study Offspring cohort

| | Training | | Test | |
|---|---|---|---|---|
| | (n = 1,280) | | (n = 639) | |
| | CHD* | No CHD† | CHD* | No CHD† |
| Gender | | | | |
| (count) | | | | |
| Male | 18 | 524 | 9 | 262 |
| Female | 10 | 728 | 5 | 363 |
| Age | | | | |
| (years) | | | | |
| Male | 70.6 ± 9.3 | 65.8 ± 8.2 | 66.1 ± 9.1 | 62.7 ± 9.0 |
| Female | 71.2 ± 10.3 | 66.3 ± 8.5 | 66.8 ± 9.0 | 64.9 ± 9.3 |
| Total | | | | |
| Cholesterol | | | | |
| (mg/dL) | | | | |
| Male | 171 ± 54 | 177 ± 32 | 161 ± 30 | 182 ± 32 |
| Female | 229 ± 40 | 199 ± 36 | 185 ± 49 | 197 ± 35 |
| HDL | | | | |
| Cholesterol | | | | |
| (mg/dL) | | | | |
| Male | 50 ± 16 | 50 ± 14 | 48 ± 12 | 51 ± 15 |
| Female | 57 ± 16 | 65 ± 19 | 60 ± 17 | 65 ± 19 |
| HbA1c | | | | |
| (%) | | | | |
| Male | 5.7 ± 0.4 | 5.7 ± 0.8 | 5.8 ± 0.8 | 5.6 ± 0.5 |
| Female | 6.0 ± 1.0 | 5.7 ± 0.5 | 5.8 ± 0.8 | 5.7 ± 0.6 |
| SBP | | | | |
| (mmHg) | | | | |
| Male | 137 ± 15 | 130 ± 17 | 136 ± 11 | 128 ± 16 |
| Female | 140 ± 19 | 129 ± 17 | 132 ± 22 | 127 ± 18 |
| DBP | | | | |
| (mmHg) | | | | |
| Male | 74 ± 11 | 76 ± 11 | 74 ± 8 | 77 ± 10 |
| Female | 80 ± 11 | 73 ± 10 | 72 ± 7 | 73 ± 10 |
| Smoker | | | | |
| (count) | | | | |
| Male | 1 (6%) | 35 (7%) | 2 (22%) | 16 (6%) |
| Female | 2 (20%) | 57 (8%) | 0 (0%) | 32 (9%) |
| Blood | | | | |
| Pressure | | | | |
| Treatment | | | | |
| (count) | | | | |
| Male | 12 (67%) | 265 (51%) | 4 (44%) | 112 (43%) |
| Female | 3 (30%) | 294 (40%) | 4 (80%) | 157 (43%) |

*Those diagnosed with CHD within three years of contributing biomaterial during the Offspring Cohort eighth examination cycle.
†Those not diagnosed with CHD within three years of contributing biomaterial during the Offspring Cohort eighth examination cycle.
HDL: high-density lipoprotein, HbA1c: Hemoglobin A1c, SBP: systolic blood pressure, DBP: diastolic blood pressure.

### Example 3—Intermountain Healthcare Cohort

The second de-identified cohort consisting of 159 subjects were drawn from the Intermountain Healthcare (IM) Heart Institute INSPIRE registry, where participants contributed biomaterial and have electronic medical records (EMR). These subjects were subjects who underwent coronary angiography at IM, provided consent to participate in the registry, and for whom both DNA from the time of the catheterization (i.e. index) and clinical follow up status with respect to incident CHD status were available. As documented in their medical records, each of the subjects had stenosis of <50% of each of their main cardiac arteries with no other clinical evidence of an atherosclerotic heart disease event prior to or at the time of their coronary angiogram. Incident CHD status was determined based on follow-up EMR data. Incident CHD was considered present if the subject was clinically diagnosed with CHD (>70% stenosis) on angiography, had a myocardial infarction, revascularization or death due to CHD within three years of index coronary angiography and biomaterial collection.

When available, conventional risk factor values (age, gender, systolic blood pressure (SBP), diastolic blood pressure (DBP), high-density lipoprotein (HDL) cholesterol level, total cholesterol level, hemoglobin Alc (HbA1c), and smoking status) also were obtained. The blood pressure values were from the admission assessment for the index coronary angiogram. For cholesterol and HbA1c, they were first available values in the 12 months prior to and 3 months after catheterization. The samples were randomly split into validation (50%) and test (50%) sets, stratified by incident CHD status, where 80 subjects (12/39 males and 11/41 females diagnosed with clinical CHD within three years of the eighth examination cycle ascertainment) were part of the validation set and 79 subjects (11/38 males and 10/41 females diagnosed with clinical CHD within three years of the eighth examination cycle ascertainment) were part of the test set. Please note that, in contrast to the FHS sample where class imbalance is evident, incident cases were intentionally selected for this cohort to ensure better balance between cases and controls. The demographics and conventional risk factors of these individuals are summarized in Table 2.

TABLE 2
Summary of demographics and conventional CHD risk factors
for the Intermountain Healthcare validation and test sets

| | Validation | | Test | |
|---|---|---|---|---|
| | (n = 80) | | (n = 79) | |
| | CHD* | No CHD† | CHD* | No CHD† |
| Gender | | | | |
| (count) | | | | |
| Male | 12 | 27 | 11 | 27 |
| Female | 11 | 30 | 10 | 31 |
| Age | | | | |
| (years) | | | | |
| Male | 61.4 ± 13.4 | 61.4 ± 15.8 | 63.9 ± 16.8 | 61.3 ± 17.4 |
| Female | 62.9 ± 16.7 | 68.5 ± 11.4 | 66.1 ± 11.8 | 63.9 ± 15.2 |
| Total | | | | |
| Cholesterol | | | | |
| (mg/dL) | | | | |
| Male | 144 ± 42 | 178 ± 40 | 169 ± 38 | 172 ± 36 |
| Female | 190 ± 49 | 183 ± 42 | 196 ± 58 | 175 ± 26 |
| HDL | | | | |
| Cholesterol | | | | |
| (mg/dL) | | | | |
| Male | 37 ± 9 | 39 ± 10 | 41 ± 11 | 38 ± 14 |
| Female | 58 ± 14 | 61 ± 22 | 52 ± 12 | 50 ± 14 |
| HbA1c | | | | |
| (%) | | | | |
| Male | 6.2 ± 1.2 | 5.9 ± 0.4 | 6.3 ± 1.3 | 6.4 ± 1.1 |
| Female | 5.9 ± 0.4 | 6.1 ± 1.4 | 6.9 ± 2.4 | 5.4 ± 0.5 |
| SBP | | | | |
| (mmHg) | | | | |
| Male | 152 ± 23 | 143 ± 22 | 143 ± 21 | 143 ± 27 |
| Female | 141 ± 18 | 152 ± 26 | 153 ± 21 | 147 ± 19 |
| DBP | | | | |
| (mmHg) | | | | |
| Male | 84 ± 11 | 85 ± 12 | 79 ± 11 | 83 ± 16 |
| Female | 75 ± 6 | 81 ± 13 | 86 ± 11 | 78 ± 12 |
| Smoker | | | | |
| (count) | | | | |
| Male | 0 (0%) | 2 (7%) | 1 (9%) | 0 (0%) |
| Female | 1 (9%) | 1 (3%) | 2 (20%) | 1 (3%) |
| Blood | | | | |
| Pressure | | | | |
| Treatment | | | | |
| (count) | | | | |
| Male | 3 (25%) | 7 (26%) | 5 (45%) | 10 (37%) |
| Female | 3 (27%) | 10 (33%) | 3 (30%) | 16 (52%) |

*Those diagnosed with CHD within three years of contributing biomaterial during the Offspring Cohort eighth examination cycle.
†Those not diagnosed with CHD within three years of contributing biomaterial during the Offspring Cohort eighth examination cycle.
HDL: high-density lipoprotein, HbA1c: Hemoglobin A1c, SBP: systolic blood pressure, DBP: diastolic blood pressure.

Genome-wide DNA methylation and genetic assessments for each of these 159 subjects were conducted by the University of Minnesota Genome Center using the Illumina Infinium MethylationEpic Beadchip array and the Illumina Infinium Multi-Ethnic Global BeadChip array (San Diego, CA, USA), respectively. These data were then subjected to the same quality control procedure described above for the FHS samples. A total of 862,593 methylation and 818,046 SNP loci survived quality control measures. For DNA methylation, loci common to both the Illumina 450K and EPIC arrays were retained, resulting in 437,242 loci for further analysis. Similarly for SNPs, those loci common to both genotyping arrays were retained, resulting in 80,371 loci for further analysis.

### Example 4—Integrated Genetic-Epigenetic Incident Coronary Heart Disease Risk Prediction Model

Because one of the aims of this study is to translate array-based methylation loci to clinically implementable digital PCR (dPCR) assays, which has fixed constraints on precision, prior to performing data mining exclusively using data from the FHS training set, the methylation variables were reduced to include loci whose delta beta (Δβ) (absolute difference between case and controls) was at least 0.03. Covariate shift (Quionero-Candela et al, 2009, Dataset Shift in Machine Learning, The MIT Press) between the FHS training set and IM validation set was used to further reduce the number of methylation loci. As a result of both of these variable reduction steps, 5,571 methylation probes remained for downstream analysis. All methylation loci beta values were converted into M-values and subsequently scaled to have zero mean and unit variance.

All data mining, feature selection, model development and model tuning were performed exclusively on the FHS training set. We integrated the 5,571 methylation loci with the 80,371 SNPs to mine for integrated genetic-epigenetic biomarkers that are highly predictive of risk for incident CHD within three years. Our data mining approach has been outlined in previous publications (Dogan et al., 2018, Genes, 9:641; Dogan et al., 2018, PLOS One, 13: e0190549). All analyses were performed in Python. Briefly, an undersampling-based approach was implemented to account for the high class imbalance and coupled to an ensemble of machine learning algorithms (Random Forest, Support Vector Machine and Logistic Regression) that incorporated cross-validation to uncover non-linear methylation-SNP interactions and highly predictive biosignatures in the FHS training set (Han et al., 2011, Data Mining: Concepts and Techniques, Elsevier). As a result, a marker set was selected consisting of three DNA methylation loci and five SNPs that had the best combined performance with respect to area under the receiver operating characteristic curve (AUC), sensitivity and specificity. The ensemble model consisting of these eight biomarkers underwent hyperparameter tuning and was finalized for testing. The final trained integrated genetic-epigenetic model was then applied on the FHS test, IM validation and IM test sets to determine the AUC, sensitivity and specificity in these sets.

To better understand if adding conventional CHD risk factors (age, gender, systolic blood pressure (SBP), diastolic blood pressure (DBP), high-density lipoprotein (HDL) cholesterol level, total cholesterol level, hemoglobin Alc (HbA1c), and smoking status) to the integrated genetic-epigenetic model could improve performance, each risk factor was added to the final trained model and tested on the FHS test, IM validation and IM test sets.

### Example 5—Polygenic Risk Score

To understand how the performance of the integrated genetic-epigenetic model described herein compared to that of Polygenic Risk Score (PRS) for incident CHD risk prediction, PRS was calculated using summary statistics from a genome-wide meta-analysis of CHD that were performed in 60,801 cases and 123,504 controls using Python Version 3.7 (Nikpay et al., 2015, Nat. Genet., 47:1121-30). Because only 80,371 SNPs overlapped between the Affymetrix array that was used to profile FHS subjects and the MultiEthnic Global BeadChip array that was used to profile IM subjects, PRS was modelled three ways.

The first was to calculate PRS based on 57,647 overlapping SNPs between both arrays that also had corresponding CHD associated log OR. For each subject, PRS was calculated by taking the product of the number of alleles associated with risk and the respective SNP's log odds ratio (log OR) for each SNP that were subsequently summed across all SNPs. Using undersampling-based logistic regression to account for class imbalance, a PRS model was fitted in the FHS training set and tested on the FHS test, IM validation and IM test sets, and the AUC, sensitivity and specificity of this model (Model 1) in each of these datasets were evaluated.

The second was to calculate PRS in the FHS cohort using 394,304 SNPs from the Affymetrix array that had corresponding CHD associated log OR. Once PRS was calculated, the same modelling approach was used to build a model in the FHS training set that was subsequently only tested on the FHS test set. The AUC, sensitivity and specificity of this model (Model 2) were evaluated.

The third was to calculate PRS in the IM cohort using 527,720 SNPs from the Illumina Multi-Ethnic Global array that had corresponding CHD associated log OR. Once PRS was calculated, the same modelling approach was used to build a model in the IM validation set that was subsequently only tested on the IM test set. The AUC, sensitivity and specificity of this model (Model 3) were evaluated.

### Example 6—Survival Analysis and Prognostic Scores

Using data from the FHS test, IM validation and IM test sets, a Kaplan-Meier survival curve was fitted to display the time to incident CHD event within three years as a function of risk group (high vs. low) as predicted by the integrated genetic-epigenetic model. The y-axis represents the probability of not having an incident CHD event within three years. The 95% confidence interval (CI) for each of the distribution was calculated and the distributions of the high and low risk groups were compared using the log-rank test.

The two risk groups then were transformed into three clinical prognostic scores (score 1=low risk, score 2=intermediate risk, score 3=high risk) using the probability of having an incident event as predicted by the integrated genetic-epigenetic model. A Kaplan-Meier survival curve was fitted for these prognosis scores alongside their respective 95% CIs and compared using the log-rank test.

### Example 7—Conventional Risk Factors-Based Model

To compare the performance of the integrated genetic-epigenetic model described herein to two commonly used conventional risk factors-based models, FRS and PCE, these risk calculators were implemented on both cohorts to identify those at high risk for CHD incidence (>20%). The variables used in this analysis include age, gender, total cholesterol, HDL, SBP, DBP, diabetes status, smoking status, and whether individuals are undergoing blood pressure treatment. Individuals with missing values and those with values outside the allowed range (e.g. for PCE, age must be between 20-79) were excluded from this analysis.

### Example 8—Digital PCR Assay Development

Array-based clinical testing can be time consuming and costly. Simple, readily available Taqman assays can be used to profile SNPs of interest from genotyping arrays. However, there are limited options for profiling methylation loci of interest for clinical tests in a timely and cost effective manner. To demonstrate that the approach described herein can be used in a clinical setting, the array-based methylation biomarkers in the integrated genetic-epigenetic model described herein were translated into dPCR assays. For each of the methylation loci, DNA from the IM cohort was bisulfite converted using the Qiagen EpiTect Bisulfite kit (Hilden, Germany). The bisulfite converted DNA was subjected to PCR amplification using custom primers. An aliquot of the amplified DNA was used to perform dPCR using custom primer and probe sets capable of distinguishing methylated and unmethylated targets. Correlation analysis was performed between the dPCR beta values and array beta values for each of the locus to demonstrate successful translation.

### Example 9—Results

The clinical and demographic characteristics of the FHS and IM cohorts is outlined in Tables 1 and 2, respectively. The average age of subjects in the FHS and IM cohorts was in the mid and early 60s, respectively, with the age range in both cohorts extending from at least the lower 40s to the upper 80s. All of the subjects from the FHS cohort were of European ancestry, but at least 10 of the subjects in the IM cohort were of non-European ancestry. The most notable difference was with respect to gender. The FHS cohort had more females than males, while the IM subjects were intentionally selected to maintain gender balance in the cohort. In general, on average, total and HDL cholesterol levels were higher in FHS compared to IM and vice versa for HbA1c, SBP and DBP. Furthermore, in both the FHS and IM cohorts, on average, for incident cases and controls, total and HDL cholesterol levels were higher in females than males.

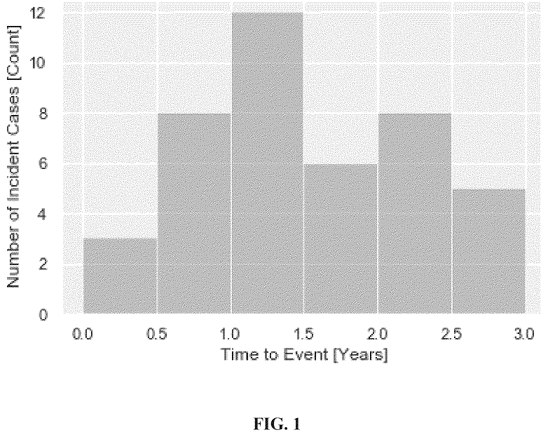

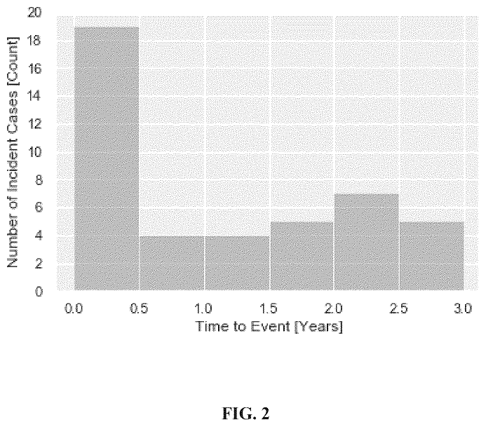

The distribution of the number of incident cases over the three year period for FHS and IM are shown in FIGS. 1 and 2, respectively. Among the 42 FHS Offspring subjects diagnosed with CHD within three years of the eighth examination cycle, the highest (29%) and lowest (7%) number of incident cases occurred between 12-18 months and 0-6 months, respectively. In contrast, among the 44 IM subjects diagnosed with CHD within three years of index coronary angiography, the highest (43%) number of subjects had their first event within 6 months of index coronary angiography, whereas the lowest (9%) number of incident cases occurred between 6-12 and 12-18 months. Still, when the entire three year incidence window was considered, the average time to event in both cohorts was similar at 1.5±0.7 and 1.1±1.0 years for FHS and IM, respectively.

### Example 10—Integrated Genetic-Epigenetic Incident Coronary Heart Disease Risk Prediction Model

Using integrated genome-wide SNP and methylation data from the 1,919 subjects in the FHS training set, an incident CHD prediction model was built to identify those at high risk of having a heart attack or sudden death within three years. This final ensemble model consisted of a total of eight biomarkers, three of which were DNA methylation biomarkers and the remaining five were SNPs. The three methylation loci are cg00300879 (TSS200 of CNKSR1), cg09552548 (Intergenic), and cg14789911 (Body of SPATCIL), while the five SNPs are rs11716050 (LOC105376934), rs6560711 (WDR37), rs3735222 (SCIN/LOC107986769), rs6820447 (intergenic), and rs9638144 (ESYT2). The integrated genetic-epigenetic model described herein performed with an AUC, sensitivity and specificity of 0.90, 0.85, and 0.75, respectively, when evaluated with the same FHS training set.

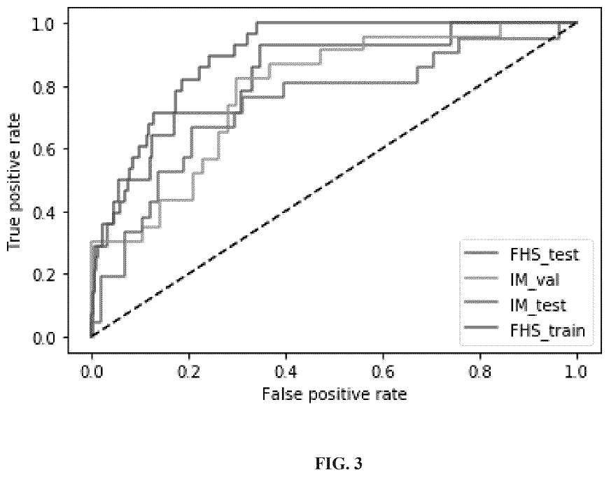

This model was then evaluated in the FHS test, IM validation and IM test sets. The AUC sensitivity and specificity of the final model in these sets are summarized in Table 3. The ROC curves are shown in FIG. 3. Briefly, the average AUC, sensitivity and specificity in these sets are 0.79, 0.76 and 0.73, respectively. These performance metrics indicated good generalizability of the trained model to the FHS test set and the external IM cohort. The performance breakdown of the integrated genetic-epigenetic model described herein then was evaluated in each set by gender. These results are summarized in Table 4. For men, the average AUC, sensitivity and specificity of the integrated genetic-epigenetic model across the FHS test, IM validation and IM test sets were 0.79, 0.75 and 0.74, respectively. For women, the average AUC, sensitivity and specificity of the integrated genetic-epigenetic model across the FHS test, IM validation and IM test sets were 0.80, 0.77 and 0.72, respectively. The similar performance metrics for both men and women and across cohorts once again indicate good generalizability of the tool.

TABLE 3
Performance of our integrated genetic-epigenetic model in the
Framingham Heart Study and Intermountain Healthcare cohorts

| Dataset | AUC | Sensitivity | Specificity |
|---|---|---|---|
| Framingham Heart Study | | | |
| Training set | 0.90 | 0.85 | 0.75 |
| Test set | 0.84 | 0.79 | 0.75 |
| Intermountain Healthcare | | | |
| Validation set | 0.78 | 0.78 | 0.74 |
| Test set | 0.74 | 0.71 | 0.71 |

AUC: area under the receiver operating characteristic curve.

TABLE 4
Performance of our integrated genetic-epigenetic model by gender in
the Framingham Heart Study and Intermountain Healthcare cohorts

| Dataset | AUC | Sensitivity | Specificity |
|---|---|---|---|
| FHS training | | | |
| Male | 0.90 | 0.89 | 0.76 |
| Female | 0.89 | 0.80 | 0.74 |
| FHS test | | | |
| Male | 0.82 | 0.78 | 0.77 |
| Female | 0.88 | 0.80 | 0.73 |
| IM validation | | | |
| Male | 0.81 | 0.75 | 0.74 |
| Female | 0.76 | 0.82 | 0.73 |
| IM test | | | |
| Male | 0.74 | 0.73 | 0.70 |
| Female | 0.75 | 0.70 | 0.71 |

FHS: Framingham Heart Study cohort, IM: Intermountain Healthcare cohort, AUC: area under the receiver operating characteristic curve.

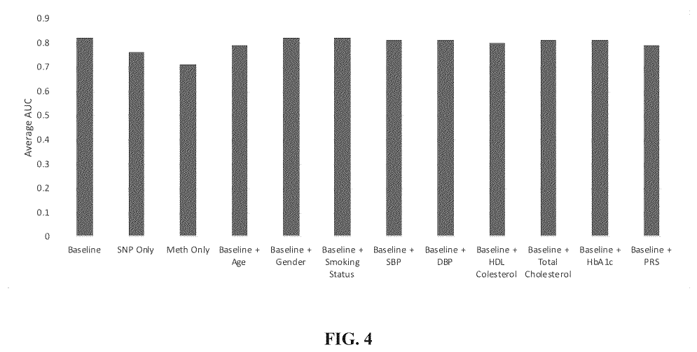

To demonstrate that the performance of the integrated genetic-epigenetic model described herein is driven by the integration of DNA methylation signatures with SNPs, models were fitted with only the five SNPs and only the three DNA methylation biomarkers. The average AUCs of these models across all four sets relative to that of the integrated genetic-epigenetic model is shown in FIG. 4. Based on these AUCs, these findings suggest that integrating both types of biomarkers allows better identification of the high and low risk groups. It was found that, on average, DNA methylation loci contributed largely to sensitivity while SNPs contribute to specificity. Similarly, to better understand if the addition of conventional CHD risk factors improves the performance of the integrated genetic-epigenetic model described herein, the average AUC of the integrated genetic-epigenetic model was compared across all four sets (baseline) to that of models that incorporated each of these risk factors. The AUCs of these models relative to the baseline also is summarized in FIG. 4. None of the additions resulted in an increase in average AUC compared to the integrated genetic-epigenetic model, suggesting that the integrated genetic-epigenetic biomarkers are capturing variance associated with conventional risk factors.

### Example 11—Polygenic Risk Score

To better understand the ability of a model that only incorporates SNPs (i.e. PRS) for incident CHD risk prediction compared to the integrated genetic-epigenetic model described herein that integrates three methylation biomarkers with five SNPs, three PRS models were trained and tested. The performance of Models 1, 2 and 3 in the various sets are summarized in Table 5. The AUC, sensitivity and specificity of these models ranged from 0.41-0.54, 0.22-0.50 and 0.47-0.69, respectively.

TABLE 5
Performance of Polygenic Risk Score models

| Model | AUC | Sensitivity | Specificity |
|---|---|---|---|
| Model 1: overlapping SNPs | | | |
| FHS test | 0.54 | 0.50 | 0.59 |
| IM validation | 0.41 | 0.22 | 0.56 |
| IM test | 0.45 | 0.38 | 0.57 |
| Model 2: FHS SNPs | | | |
| FHS test | 0.41 | 0.43 | 0.47 |
| Model 3: IM SNPs | | | |
| IM test | 0.52 | 0.38 | 0.69 |

FHS: Framingham Heart Study cohort, IM: Intermountain Healthcare cohort AUC: area under the receiver operating characteristic curve.

These findings indicate that all three versions, on average, had better specificity than sensitivity. Model 1, which was trained on the FHS training set with the least number of SNPs compared to Models 2 and 3, performed with the best AUC and sensitivity on the FHS test set of 0.54 and 0.50, respectively. However, the highest specificity of 0.69 was observed for Model 3, which was trained on the IM validation set with the most number of SNPs and tested on the IM test set. It was found that the models were not highly generalizable between cohorts.

The AUC, sensitivity and specificity of the integrated genetic-epigenetic model clearly outperformed that of PRS. The approach described herein also is more generalizable, consisted of far fewer SNPs and incorporated informative DNA methylation biomarkers in addition to SNPs. However, to determine whether the addition of PRS to the integrated genetic-epigenetic model could potentially improve risk assessment, the average AUC of the integrated genetic-epigenetic model described herein (baseline) was compared with and without incorporating Model 1 PRS. The average AUC when PRS was incorporated is 0.79, which is lower than the average AUC of the baseline model as shown in FIG. 4.

### Example 12—Survival Curve and Prognostic Scores

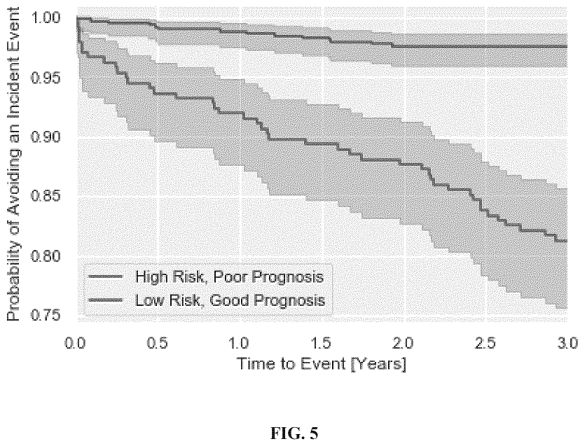

The Kaplan-Meier survival curve for the high and low risk groups is shown in FIG. 5. For those with poor prognosis (i.e. at higher risk of having an incident CHD event within three years), there is a clear rapid drop in probability of not having an incident even compared to the good prognosis group (i.e. at lower risk of having an incident CHD event within three years). The log-rank test p-value between these two groups of 2.46e-16 indicate a statistically significant difference between their distributions.

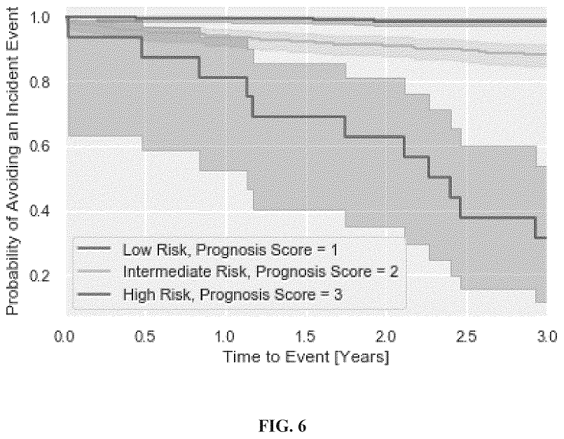

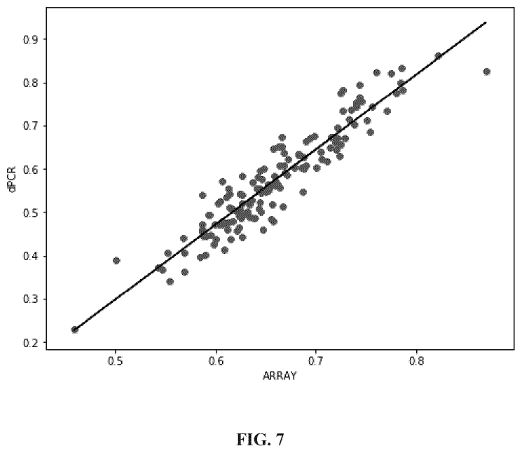

These groups then were translated into clinical prognostic score of 1, 2 and 3 to indicate low, intermediate and high risk groups, respectively. The Kaplan-Meier survival score for these score are shown in FIG. 6. Once again, there is a clear rapid drop in probability of not having an incident event within three years with a high prognostic score. The log-rank test p-value between these groups is 9.39e-33, indicating a statistically significant difference between their distributions. For the high risk group with a prognostic score of 3, the positive predictive value (PPV) is 69%. For the low risk group with a prognostics score of 1, the negative predictive value (NPV) is 99%. The intermediate risk group with a prognostic score of 2 has PPV and NPV of 15% and 94%, respectively. Thus, individuals in the high and intermediate risk groups are 50 and 10 times more likely to have an incident CHD event in the next three years, respectively, compared to the low risk group.

### Example 13—Conventional Risk Factors-Based Model

To compare the performance of the approach described herein to commonly used standard risk factors-based calculators, FRS and PCE, these estimators were applied to both cohorts. A majority of the risk factors considered by both of these risk calculators are the same (age, sex, smoking status, diabetes, SBP, total cholesterol and HDL cholesterol). In addition to these risk factors, the FRS calculator considers DBP, whereas the PCE calculator considers the use of blood pressure treatment. Due to missing values and constraints in these calculators such as with respect to age, not all subjects were evaluated. The performance of these models across both cohorts are summarized in Table 6, and the breakdown by gender is summarized in Table 7. On average, FRS performed with 0.23 and 0.91 sensitivity and specificity, respectively. The PCE risk estimator, on average, performed with a sensitivity and specificity of 0.55 and 0.65, respectively. The FRS calculator had better specificity over sensitivity in both cohorts and vice versa for the PCE calculator. Similarly, for the gender breakdown, in general, FRS tended to perform with better specificity for both men and women, while PCE tended to perform better with respect to sensitivity. The integrated genetic-epigenetic approach was 52% and 51% more sensitive for men and women, respectively, compared to FRS. It was also 10% and 39% more sensitive and 10% and 6% more specific for men and women, respectively, compared to PCE.

TABLE 6
Performance of the Framingham Risk Score and ASCVD Pooled
Cohort risk estimators in the FHS and IM cohorts.

| Risk Estimator | Sensitivity | Specificity |
|---|---|---|
| Framingham | | |
| Risk Score | | |
| FHS cohort | 0.15 | 0.93 |
| IM cohort | 0.31 | 0.89 |
| ASCVD Pooled | | |
| Cohort Equation | | |
| FHS cohort | 0.41 | 0.74 |
| IM cohort | 0.69 | 0.55 |

FHS: Framingham Heart Study cohort, IM: Intermountain Healthcare cohort.

TABLE 7
Performance of the Framingham Risk Score and ASCVD Pooled
Cohort risk estimators in the FHS and IM cohorts by gender

| Risk Estimator | Sensitivity | Specificity |
|---|---|---|
| Framingham | | |
| Risk Score | | |
| FHS cohort | | |
| Male | 0.12 | 0.86 |
| Female | 0.22 | 0.98 |
| IM cohort | | |
| Male | 0.33 | 0.85 |
| Female | 0.29 | 0.93 |
| ASCVD Pooled | | |
| Cohort Equation | | |
| FHS cohort | | |
| Male | 0.52 | 0.66 |
| Female | 0.18 | 0.81 |
| IM cohort | | |
| Male | 0.78 | 0.61 |
| Female | 0.57 | 0.50 |

FHS: Framingham Heart Study cohort, IM: Intermountain Healthcare cohort.

### Example 14—Digital PCR Assays

Because one of the goals of this study is to demonstrate the applicability of the integrated genetic-epigenetic model described herein in conventional clinical or research settings, the time consuming, labor intensive genome-wide methylation approach was translated into a simple, quick to perform methylation sensitive dPCR assays for methylation loci included in the final model. As an example, in FIG. 5, the translation of cg00300879 is shown. The Pearson correlation between methylation values as determined by dPCR to that of their corresponding array values for cg00300879 is 0.94. This high correlation suggests that dPCR is a viable alternative to array-based DNA methylation assessments.

### Example 15—Additional Data

Appendix A shows a list of CpGs whose methylation is associated with CVD. Appendix B shows a list of genes whose methylation is associated with CVD. Appendix C shows a list of SNPs associated with CVD. The numerical values provided in Appendix A, B, and C are the mean of 5-fold cross validation scores, AUC ROC (Area Under The Receiver Operating Characteristic Curve), sensitivity and specificity, which were computed by the diabetes assessment/prediction algorithm described herein. Sensitivity is the true positive rate and specificity is the true negative rate.

### Example 16—Materials and Methods

### Study Approval:

All subjects who participated in the study provided informed written consent. All procedures used in the study were approved by the University of Iowa Institutional Review Board (IRB201706713).

### Study Participants:

The 39 participants whose data are included in this study were part of a cohort of 67 subjects recruited in a series of advertisements seeking adult daily smokers, distributed to subjects and staff at the University of Iowa Hospitals and Clinics. Those subjects who were potentially interested in the study were invited to complete an online survey on their smoking habits. Those subjects who reported smoking more than 10 cigarettes a day and had at least 5 pack-years of lifetime consumption in the survey were then invited to participate in the smoking cessation protocol.

In brief, as part of the study to determine the effects of smoking cessation on pulmonary inflammation, subjects were offered USD $400 if they successfully quit smoking. Successful quitting was defined as a self-report of quitting smoking accompanied by serum cotinine values of less than 10 ng/mL at the first-, second-, and third-monthly clinical visit. Subjects were encouraged to stop “cold turkey” and to abstain from using standard smoking cessation treatments, particularly nicotine replacement therapy, to quit smoking. Subjects were offered a brief counseling session led by a research assistant at each study visit and a weekly phone call over the first month of the study. Subjects were considered treatment failures if they had serum cotinine values above 10 ng/mL at any time point or failed to attend any of the clinical visits. Only 20 of the original 67 subjects completed all procedures, reported quitting smoking, and had serum cotinine values of <10 ng/ml at all three monthly clinic visits. Nineteen others who provided DNA for this study also completed all four visits but had serum cotinine values of >10 ng/ml at one or more visits.

### Laboratory Measures:

All subjects were phlebotomized at intake and during each monthly clinic visit to provide serum and DNA for the current study. Serum cotinine levels were determined by University of Iowa Diagnostic Laboratories under standard CLIA-compliant procedures. Relative change in DNA methylation at cg05575921, a well-established epigenetic indicator of smoking intensity, and three other sites used in the Epi+ Gen CHD™ test were quantified by personnel blind to subject status. Whole blood DNA from each subject at each time point (monthly meeting) was prepared as previously described (Philibert et al., 2020, Am. J. Med. Genet. Part B Neuropsychiatr. Genet., 183:51-60).

DNA methylation at cg05575921 and the three methylation sites in the Epi+ Gen CHD™ test (cg00300879, cg09552548, and cg14789911) was performed as previously described using proprietary methylation-sensitive, nested, digital primer probe sets from Behavioral Diagnostics and Cardio Diagnostics (Coralville, IA, USA) and droplet digital PCR reagents and machinery from Bio Rad (Carlsbad, CA, USA). In brief, 1 μg of DNA from each subject at study intake (baseline) and study exit (month 3) was bisulfite-converted using a Fast 96 Epitect Kit (Qiagen, Germany), with the resulting DNA being eluted using 70 μL of 10 mM Tris buffer (pH 8.0). A 3 μL aliquot of the resulting product was pre-amplified using the assay-specific pre-amplification mix, then diluted 1:1500 for the Epi+ Gen CHD™ assay, or 1:3000 for the cg05575921 assay. After dilution, a 5 uL aliquot containing approximately 10,000 amplicons—mixed with universal droplet digital PCR reagents and fluorescent primer probe sets specific to the cg00300879, cg09552548, cg14789911, and cg05575921 loci—was partitioned into droplets and then PCR amplified using a QX-200 Droplet Digital PCR system (Bio Rad) according to manufacturer's instructions. After amplification was complete, the number of droplets containing amplicons with at least one “C” allele, one “T” allele, or neither allele was then determined using a Bio-Rad QX-200 Droplet Reader, and the absolute ratio of methylated to total CpG methylation at each was determined by the proprietary Bio Rad Quantisoft™M software.

### Statistical Analyses:

All data were analyzed using the JMP Version 14 (SAS Institute, Cary, SC) using standard general linear model equations (Andersen et al., 2021, Epigenetics, 1-13). Group comparisons of continuous variables were compared using T-Tests. Bivariate regression was used to analyze the relationship of changes of epigenetic-indicated smoking intensity (cg05575921) to change in cardiac methylation marker (cg00300879, cg09552548, and cg14789911) status.

### Example 17—Results

The clinical and demographic characteristics of the 39 subjects who completed all four clinic visits and whose data were used in this study are given in Table 8. In brief, they tended to be in the late 30s to early 40s in age, with a slight majority being male. All but two of the subjects were White.

TABLE 8
Demographic and Clinical Characteristics of the Subjects

| | Quitters | Non-quitters |
|---|---|---|
| N | 20 | 19 |
| Age | 39.8 ± 9.9 | 45 ± 10.2 |
| Gender | | |
| Male | 11 | 11 |
| Female | 9 | 8 |
| Ethnicity | | |
| White | 20 | 17 |
| African American | — | 1 |
| Other | — | 1 |
| Pack-Year Consumption | 22 ± 9.6 | 34 ± 25 |
| Cigarettes per day | 16 ± 6 | 19 ± 13 |
| Intake Cotinine (ng/mL) | 206 ± 93 | 278 ± 135 |
| Intake Methylation (%) | | |
| cg00300879 | 52.9 ± 9.1 | 58.3 ± 11.7 |
| cg09552548 | 30.0 ± 11.7 | 32.0 ± 13.4 |
| cg14789911** | 89.7 ± 9.3 | 81.2 ± 16.4 |
| cg05575921 | 57.1 ± 22.1 | 47.2 ± 18.3 |
| Delta Methylation (%) | | |
| over 90 days | | |
| cg00300879 | −0.7 ± 1.6 | 1.0 ± 4.6 |
| cg09552548 | 0.1 ± 0.8 | 0.2 ± 1.0 |
| cg14789911** | −1.5 ± 3.4 | −1.1 ± 5.2 |
| cg05575921 | −7.6 ± 5.8 | −2.1 ± 5.5 |

**nominally different at p < 0.05

Only 20 of the subjects who completed all four clinic visits managed to quit smoking, as evidenced by negative cotinine values at all three monthly visits. There were no significant differences in cigarette consumption or serum cotinine values between those who quit and those who did not quit (p >0.05 for both).

We determined methylation levels at each of the three CpG sites used in the Epi+ Gen CHID™ test in each of the subjects at study entry and study exit ninety days later (see Table 8). Please note that because the set point of these methylation sites is genetically contextual and we did not determine genotype at the five sites used in this test, a direct comparison of the methylation values for those who quit versus those who did not quit is not possible. Nevertheless, in general, lower methylation values at cg00300879 and cg09552548, but higher methylation levels at cg14789911, are associated with increased risk for incident CHD within three years (Dogan et al., 2021, Epigenomics, 13 (14): 1095-112).

Over the course of the study, methylation arithmetically increased at cg00300879 and cg09552548 and decreased at cg14789911 in those who quit smoking. However, using a categorical approach to classify smoking cessation, none of the changes at these three loci were significant. The subjects who were unsuccessful in quitting smoking manifested lesser degrees of change at the three loci over the 90-day period, all of which were also not significantly associated with categorical quitting status.

However, when considering these results, it is important to realize that, just as not all cases of CHD are equally severe, not all smokers consume the same number of cigarettes. Fortunately, the use of cg05575921 as a metric for change in smoking intensity permits the stable objective measurement of smoking intensity. Recently, we have shown that changes in cg05575921 methylation in response to smoking cessation are also dose-dependent. Therefore, in order to determine whether the changes in smoking intensity were related to the changes in methylation, we analyzed the relationship of the change of methylation at each of the cardiac markers to the change in smoking intensity as measured by change in cg05575921.

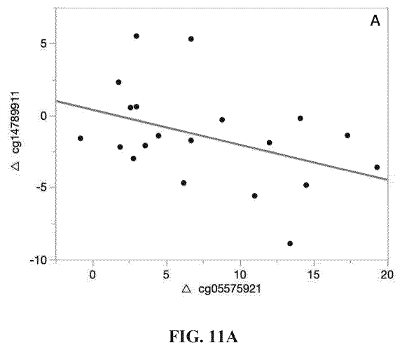

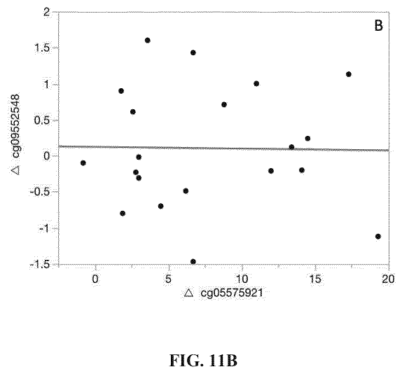

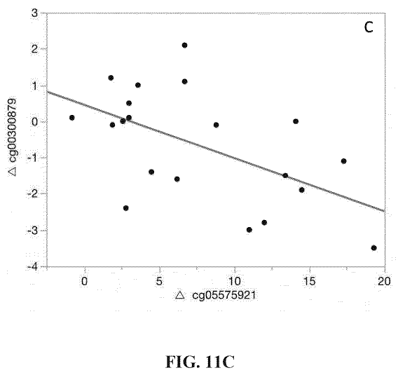

As expected, even though some of the subjects showed objective evidence of decreasing the rate of smoking, there were no significant relationships between the change in smoking intensity and changes in methylation at any of the three cardiac-specific loci in the 19 subjects who did not completely quit smoking. However, in the group of 20 subjects who managed to quit smoking completely, after correction for multiple comparisons, there was a significant relationship between the smoking-cessation-induced reversion of methylation at cg05575921 to an increase in methylation at cg00300879 (Adj R2 0.26, p<0.04; FIG. 11C), with the changes in methylation at cg147989911 failing to achieve statistical significance (Adj R2 0.14, p<0.07; FIG. 11A).

The current findings have potential for improving CVD or CHD prevention in those with multiple risk factors. In particular, we believe that developing an epigenetic method of monitoring CVD and CHD risk may improve management of those with elevated cholesterol levels and subclinical or overt type 2 diabetes. A conundrum for clinicians is the knowledge that statin-induced decreases in serum cholesterol levels are often associated with increases in HbA1c levels. Overall, the risk/benefit ratio for the use of statins is favorable. Whether this general benefit applies to all patients equally is not known, because current algorithms cannot simultaneously consider changes in lipid and HbA1c levels. However, because each of the methylation markers maps differently to principle components of the methylation response associated with CVD and CHD, the change in overall risk as a consequence of changes in serum cholesterol and HbA1c levels can be assessed simultaneously by the integrated assessment tools described herein. For most patients, the added information obtained by retesting methylation levels is unlikely to significantly change risk management. However, for some patients, particularly those with genetic polymorphisms that alter HbA1c levels, the added information could be valuable. In summary, the current results of this study show changes at the CpG loci predictive of incident CHD in association with the biochemically verified treatment of a risk factor for CHD.

### Example 18—Addition of Protein Biomarkers to SNP-CpG Biomarkers

At least one protein biomarker is added to a method employing a biomarker scoring system with at least one SNP and/or one methylation biomarker that offers, among other things, an improvement in the ability of the biomarker scoring system to diagnose or prognose cardiovascular disease. These experiments rely on the subjects from the Framingham Heart Study Offspring Cohort described herein. The subjects selected for this analysis consist of those that have data on at least one protein biomarker included below in Table 9 and have information on one or more types of CVD.

TABLE 9
Representative List of Protein Biomarkers

Adiponectin
Alpha-1-Antitrypsin (AAT)
Alpha-2-Macroglobulin (A2Macro)
Angiopoietin-1 (ANG-1)
Angiotensin-Converting 1 Enzyme (ACE)
Apolipoprotein(a) (Lp(a))
Apolipoprotein A-I (Apo A-I)
Apolipoprotein A-II (Apo A-II)
Apolipoprotein B (Apo B)
Apolipoprotein C-I (Apo C-I)
Apolipoprotein C-III (Apo C-III)
Apolipoprotein H (Apo H)
Beta-2-Microglobulin (B2M)
Brain-Derived Neurotrophic Factor (BDNF)
C-Reactive Protein (CRP)
Carbonic anhydrase 9 (CA-9)
Carcinoembryonic antigen-related cell
adhesion molecule 1 (CEACAM1)
CD5 Antigen-like (CD5L)
Decorin
E-Selectin
EN-RAGE
Eotaxin-1
Factor VII
Ferritin (FRTN)
Fetuin-A
Fibrinogen
Follicle-Stimulating Hormone (FSH)
Growth Hormone (GH)
Haptoglobin
Immunoglobulin A (IgA)
Immunoglobulin M (IgM)
Insulin
Intercellular Adhesion
Molecule 1 (ICAM-1)
Interferon gamma Induced Protein 10 (IP-10)
Interleukin-1 receptor antagonist (IL-1ra)
Interleukin-6 receptor (IL-6r)
Interleukin-8 (IL-8)
Interleukin-12 Subunit p40 (IL-12p40)
Interleukin-15 (IL-15)
Interleukin-18 (IL-18)
Interleukin-18-binding protein (IL-18bp)
Interleukin-23 (IL-23)
Kidney Injury Molecule-1 (KIM-1)
Leptin
Luteinizing Hormone (LH)
Macrophage Colony-Stimulating Factor 1 (M-CSF)
Macrophage Inflammatory Protein-1 beta (MIP-1 beta)
Matrix Metalloproteinase-2 (MMP-2)
Matrix Metalloproteinase-3 (MMP-3)
Matrix Metalloproteinase-7 (MMP-7)
Matrix Metalloproteinase-9 (MMP-9)
Matrix Metalloproteinase-9, total (MMP-9, total)
Midkine
Monocyte Chemotactic Protein 1 (MCP-1)
Monocyte Chemotactic Protein 2 (MCP-2)
Monocyte Chemotactic Protein 4 (MCP-4)
Monokine Induced by Gamma Interferon (MIG)
Myeloid Progenitor Inhibitory Factor 1 (MPIF-1)
Myoglobin
N-terminal prohormone of brain natriuretic
peptide (NT proBNP)
Osteopontin
Pancreatic Polypeptide (PPP)
Plasminogen Activator Inhibitor 1 (PAI-1)
Platelet endothelial cell adhesion molecule (PECAM-1)
Prolactin (PRL)
Pulmonary and Activation-Regulated Chemokine (PARC)
Pulmonary surfactant-associated protein D (SP-D)
Resistin
Serotransferrin (Transferrin)
Serum Amyloid P-Component (SAP)
Stem Cell Factor (SCF)
T-Cell-Specific Protein RANTES (RANTES)
Tamm-Horsfall Urinary Glycoprotein (THP)
Thrombomodulin (TM)
Thrombospondin-1
Thyroid-Stimulating Hormone (TSH)
Thyroxine-Binding Globulin (TBG)
Tissue Inhibitor of Metalloproteinases 1 (TIMP-1)
Transthyretin (TTR)
Troponin
Tumor necrosis factor receptor 2 (TNFR2)
Vascular Cell Adhesion Molecule-1 (VCAM-1)
Vascular Endothelial Growth (VEGF)
Vitamin D-Binding Protein (VDBP)
Vitamin K-Dependent Protein S (VKDPS)
Vitronectin von Willebrand Factor (vWF)

Machine learning methods such as ones described herein are used to identify at least one protein biomarker that, when added to the SNP and/or methylation biomarker scoring system, improve the predictive capability of the biomarker scoring system. To achieve this, subjects are split into training and test sets. The training set is used to identify the protein biomarker(s) and to quantify performance. The test set is used to verify the improvement in performance. The AUC, sensitivity, specificity and accuracy are quantified.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A kit for determining methylation status of at least one CpG dinucleotide and a genotype of at least one single-nucleotide polymorphism (SNP), the kit comprising:
at least one first nucleic acid primer at least 8 nucleotides in length that is complementary to a bisulfite-converted nucleic acid sequence comprising a first CpG dinucleotide at a GC locus selected from the group consisting of cg00300879, cg09552548, and cg14789911 or at a second CpG dinucleotide in linkage disequilibrium with the first CpG dinucleotide at a GC locus selected from the group consisting of cg00300879, cg09552548, and cg14789911, wherein the linkage disequilibrium has a value of R>0.3, wherein the at least one first nucleic acid primer detects a methylated or unmethylated CpG dinucleotide, and
at least one second nucleic acid primer at least 8 nucleotides in length that is complementary to a DNA sequence or a bisulfite-converted DNA sequence of a first SNP selected from the group consisting of rs11716050, rs6560711, rs3735222, rs6820447, and rs9638144 or a second SNP in linkage disequilibrium with the first SNP selected from the group consisting of rs11716050, rs6560711, rs3735222, rs6820447, and rs9638144, wherein the linkage disequilibrium has a value of R>0.3.
